SEQUENCE LISTING (49 sequences)

SEQ ID NO: 1 - protein, 13 aa, Artificial Sequence
  capsid polypeptide
DPIVMIDNDKPIT

SEQ ID NO: 2 - protein, 735 aa, Adeno-Associated Virus Type 2 [sequence omitted]

SEQ ID NO: 3 - protein, 13 aa, Artificial Sequence
  capsid polypeptide
APIVMIDNDKPIT

SEQ ID NO: 4 - protein, 13 aa, Artificial Sequence
  capsid polypeptide
DHIVMIDNDKPIT

SEQ ID NO: 5 - protein, 13 aa, Artificial Sequence
  capsid polypeptide
DPIVMVDNDKPIT

SEQ ID NO: 6 - protein, 13 aa, Artificial Sequence
  capsid polypeptide
DPIVMIDADKPIT

SEQ ID NO: 7 - protein, 13 aa, Artificial Sequence
  capsid polypeptide
DPIVMIDNYKPIT

SEQ ID NO: 8 - protein, 13 aa, Artificial Sequence
  peptide tag
AHIVMVDAYKPTK

SEQ ID NO: 9 - protein, 12 aa, Artificial Sequence
  peptide tag
KLGDIEFIKVNK

SEQ ID NO: 10 - protein, 83 aa, Artificial Sequence
  binding moiety that can form a covalent bond with a capsid polypeptide
  MOD_RES (44)..(44): Xaa = D or V
DSTTHVKFSKRDINGKELAGAMIELRNLSGQTIQSWVSDGTVKXFYLMPGTYQFVETAAPEGYELAAPITFTIDEKGQIWVDS

SEQ ID NO: 11 - protein, 83 aa, Artificial Sequence
  binding moiety that can form a covalent bond with a capsid polypeptide
  misc_feature (6)..(6): Xaa can be any naturally occurring amino acid
  misc_feature (16)..(16): Xaa can be any naturally occurring amino acid
  misc_feature (22)..(22): Xaa can be any naturally occurring amino acid
  misc_feature (28)..(28): Xaa can be any naturally occurring amino acid
  misc_feature (31)..(31): Xaa can be any naturally occurring amino acid
  misc_feature (37)..(37): Xaa can be any naturally occurring amino acid
  misc_feature (41)..(41): Xaa can be any naturally occurring amino acid
  misc_feature (51)..(51): Xaa can be any naturally occurring amino acid
  misc_feature (66)..(66): Xaa can be any naturally occurring amino acid
  misc_feature (76)..(76): Xaa can be any naturally occurring amino acid
  misc_feature (82)..(82): Xaa can be any naturally occurring amino acid
DSTTHXKFSKRDINGXELAGAXIELRNXSGXTIQSWXSDGXVKDFYLMPGXYQFVETAAPEGYELXAPITFTIDEXGQIWVXS

SEQ ID NO: 12 - protein, 123 aa, Artificial Sequence
  binding moiety that can form a covalent bond with a capsid polypeptide
  MOD_RES (65)..(65): Xaa = D or V
IDTMSGLSGETGQSGNTTIEEDSTTHVKFSKRDINGKELAGAMIELRNLSGQTIQSWVSDGTVKXFYLMPGTYQFVETAAPEGYELAAPITFTIDEKGQIWVDSTLIVGDDPIVMIDNDKPIT

SEQ ID NO: 13 - protein, 123 aa, Artificial Sequence
  binding moiety that can form a covalent bond with a capsid polypeptide
  misc_feature (4)..(4): Xaa can be any naturally occurring amino acid
  misc_feature (9)..(10): Xaa can be any naturally occurring amino acid
  misc_feature (12)..(13): Xaa can be any naturally occurring amino acid
  misc_feature (15)..(15): Xaa can be any naturally occurring amino acid
  misc_feature (19)..(19): Xaa can be any naturally occurring amino acid
  misc_feature (27)..(27): Xaa can be any naturally occurring amino acid
  misc_feature (37)..(37): Xaa can be any naturally occurring amino acid
  misc_feature (43)..(43): Xaa can be any naturally occurring amino acid
  misc_feature (49)..(49): Xaa can be any naturally occurring amino acid
  misc_feature (52)..(52): Xaa can be any naturally occurring amino acid
  misc_feature (58)..(58): Xaa can be any naturally occurring amino acid
  misc_feature (62)..(62): Xaa can be any naturally occurring amino acid
  MOD_RES (65)..(65): Xaa = D or V
  misc_feature (72)..(72): Xaa can be any naturally occurring amino acid
  misc_feature (87)..(87): Xaa can be any naturally occurring amino acid
  misc_feature (97)..(97): Xaa can be any naturally occurring amino acid
  misc_feature (103)..(103): Xaa can be any naturally occurring amino acid
  misc_feature (105)..(105): Xaa can be any naturally occurring amino acid
  misc_feature (113)..(113): Xaa can be any naturally occurring amino acid
IDTXSGLSXXTXXSXNTTXEEDSTTHXKFSKRDINGXELAGAXIELRNXSGXTIQSWXSDGXVKXFYLMPGXYQFVETAAPEGYELXAPITFTIDEXGQIWVXSXLIVGDDPXVMIDNDKPIT

SEQ ID NO: 14 - protein, 111 aa, Artificial Sequence
  binding moiety that can form a covalent bond with a capsid polypeptide
  MOD_RES (64)..(64): Xaa = D or V
DTMSGLSGETGQSGNTTIEEDSTTHVKFSKRDINGKELAGAMIELRNLSGQTIQSWVSDGTVKXFYLMPGTYQFVETAAPEGYELAAPITFTIDEKGQIWVDSTLIVGDDP

SEQ ID NO: 15 - protein, 103 aa, Artificial Sequence
  binding moiety that can form a covalent bond with a capsid polypeptide
  MOD_RES (64)..(64): Xaa = D or V
DTMSGLSGETGQSGNTTIEEDSTTHVKFSKRDINGKELAGAMIELRNLSGQTIQSWVSDGTVKXFYLMPGTYQFVETAAPEGYELAAPITFTIDEKGQIWVDS

SEQ ID NO: 16 - protein, 102 aa, Artificial Sequence
  binding moiety that can form a covalent bond with a capsid polypeptide
  MOD_RES (44)..(44): Xaa = D or V
DSTTHVKFSKRDINGKELAGAMIELRNLSGQTIQSWVSDGTVKXFYLMPGTYQFVETAAPEGYELAAPITFTIDEKGQIWVDSTLIVGDDPIVMIDNDKPIT

SEQ ID NO: 17 - protein, 122 aa, Artificial Sequence
  binding moiety that can form a covalent bond with a capsid polypeptide
  MOD_RES (64)..(64): Xaa = D or V
DTMSGLSGETGQSGNTTIEEDSTTHVKFSKRDINGKELAGAMIELRNLSGQTIQSWVSDGTVKXFYLMPGTYQFVETAAPEGYELAAPITFTIDEKGQIWVDSTLIVGDDPIVMIDNDKPIT

SEQ ID NO: 18 - protein, 92 aa, Artificial Sequence
  binding moiety for peptide tag
DSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 19 - protein, 116 aa, Artificial Sequence
  binding moiety for peptide tag
GSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 20 - protein, 7 aa, Artificial Sequence
  protease cleavage site
ENLYFQG

SEQ ID NO: 21 - protein, 7 aa, Artificial Sequence
  protease cleavage site
  MOD_RES (6)..(6): Xaa = Q or G
LEVLFXP

SEQ ID NO: 22 - protein, 6 aa, Artificial Sequence
  protease cleavage site
LVPRGS

SEQ ID NO: 23 - protein, 4 aa, Artificial Sequence
  protease cleavage site
  MOD_RES (2)..(2): Xaa = E or P
IXGR

SEQ ID NO: 24 - protein, 5 aa, Artificial Sequence
  protease cleavage site
DDDDK

SEQ ID NO: 25 - protein, 8 aa, Artificial Sequence
  protease cleavage site
LVPWELQE

SEQ ID NO: 26 - protein, 83 aa, Artificial Sequence
  binding moiety that can form a covalent bond with a capsid polypeptide
  MOD_RES (6)..(6): Xaa = valine, alanine, isoleucine, or leucine
  MOD_RES (16)..(16): Xaa = lysine, arginine, histidine, or serine
  MOD_RES (22)..(22): Xaa = methionine, leucine, phenylalanine, or tryptophan
  MOD_RES (28)..(28): Xaa = leucine, methionine, isoleucine, or tyrosine
  MOD_RES (31)..(31): Xaa = glutamine, serine, threonine, or asparagine
  MOD_RES (37)..(37): Xaa = isoleucine, leucine, methionine, or phenylalanine
  MOD_RES (41)..(41): Xaa = threonine, serine, valine, or asparagine
  MOD_RES (44)..(44): Xaa = D or V
  MOD_RES (51)..(51): Xaa = threonine, serine, valine, or asparagine
  MOD_RES (66)..(66): Xaa = alanine, isoleucine, leucine, or phenylalanine
  MOD_RES (76)..(76): Xaa = lysine, arginine, histidine, or serine
  MOD_RES (82)..(82): Xaa = aspartic acid, glutamic acid, or lysine
DSTTHXKFSKRDINGXELAGAXIELRNXSGXTIQSWXSDGXVKXFYLMPGXYQFVETAAPEGYELXAPITFTIDEXGQIWVXS

SEQ ID NO: 27 - protein, 123 aa, Artificial Sequence; identical to SEQ ID NO: 13
  binding moiety that can form a covalent bond with a capsid polypeptide
  MOD_RES (4)..(4): Xaa = methionine, isoleucine, leucine, or phenylalanine
  MOD_RES (9)..(9): Xaa = glycine, proline, cysteine, or serine
  MOD_RES (10)..(10): Xaa = glutamic acid, aspartic acid, arginine, or lysine
  MOD_RES (12)..(12): Xaa = glycine, proline, cysteine, or serine
  MOD_RES (13)..(13): Xaa = glutamine, serine, threonine, or asparagine
  MOD_RES (15)..(15): Xaa = glycine, proline, cysteine, or serine
  MOD_RES (19)..(19): Xaa = isoleucine, leucine, methionine, or phenylalanine
  MOD_RES (27)..(27): Xaa = valine, alanine, isoleucine, or leucine
  MOD_RES (37)..(37): Xaa = lysine, arginine, histidine, or serine
  MOD_RES (43)..(43): Xaa = methionine, leucine, phenylalanine, or tryptophan
  MOD_RES (49)..(49): Xaa = leucine, methionine, isoleucine, or tyrosine
  MOD_RES (52)..(52): Xaa = glutamine, serine, threonine, or asparagine
  MOD_RES (58)..(58): Xaa = isoleucine, leucine, methionine, or phenylalanine
  MOD_RES (62)..(62): Xaa = threonine, serine, valine, or asparagine
  MOD_RES (65)..(65): Xaa = D or V
  MOD_RES (72)..(72): Xaa = threonine, serine, valine, or asparagine
  MOD_RES (87)..(87): Xaa = alanine, isoleucine, leucine, or phenylalanine
  MOD_RES (97)..(97): Xaa = lysine, arginine, histidine, or serine
  MOD_RES (103)..(103): Xaa = aspartic acid, glutamic acid, or lysine
  MOD_RES (105)..(105): Xaa = threonine, serine, valine, or asparagine
  MOD_RES (113)..(113): Xaa = isoleucine, leucine, methionine, or phenylalanine

SEQ ID NO: 28 - DNA, 21 bp, Artificial Sequence
  oligonucleotide primer
agacaagcagctaccgcagat

SEQ ID NO: 29 - DNA, 20 bp, Artificial Sequence
  oligonucleotide primer
gttgcctctctggaggttgg

SEQ ID NO: 30 - DNA, 48 bp, Artificial Sequence
  oligonucleotide amplicon sequence
ctagcagaccctattgtaatgattgataatgacaagccgattactgct

SEQ ID NO: 31 - DNA, 21 bp, Artificial Sequence
  oligonucleotide primer
agacaagcagctaccgcagat

SEQ ID NO: 32 - DNA, 20 bp, Artificial Sequence
  oligonucleotide primer
gttgcctctctggaggttgg

SEQ ID NO: 33 - DNA, 54 bp, Artificial Sequence
  oligonucleotide amplicon sequence
accggtgaccctattgtaatgattgataatgacaagccgattactggcttaagt

SEQ ID NO: 34 - DNA, 69 bp, Artificial Sequence
  oligonucleotide amplicon sequence
ctagcagagaacctctatttccagggagcccacatcgtgatggtggacgcctacaagccgacgaaggct

SEQ ID NO: 35 - DNA, 75 bp, Artificial Sequence
  oligonucleotide amplicon sequence
accggtgagaacctctatttccagggagcccacatcgtgatggtggacgcctacaagccgacgaagggcttaagt

SEQ ID NO: 36 - protein, 13 aa, Artificial Sequence
  capsid polypeptide
DPIVMIDNDKPYT

SEQ ID NO: 37 - protein, 13 aa, Artificial Sequence
  capsid polypeptide
DPIVMIDNDKPIT

SEQ ID NO: 38 - protein, 17 aa, Artificial Sequence
  capsid polypeptide
GSDPIVMIDNDKPITGS

SEQ ID NO: 39 - protein, 12 aa, Artificial Sequence
  capsid polypeptide
PIVMIDNDKPIT

SEQ ID NO: 40 - protein, 11 aa, Artificial Sequence
  capsid polypeptide
PIVMIDNDKPI

SEQ ID NO: 41 - protein, 16 aa, Artificial Sequence
  peptide linker
SGGGGSGGGGSGGGGS

SEQ ID NO: 42 - protein, 15 aa, Artificial Sequence
  peptide linker
LAEAAAKEAAAKAAA

SEQ ID NO: 43 - protein, 54 aa, Artificial Sequence
  peptide linker
GSGGGSGGGSPANLKALEAQKQKEQRQAAEELANAKKLKEQLEKGSGGGSGGGS

SEQ ID NO: 44 - protein, 14 aa, Artificial Sequence
  peptide tag
VPTIVMVDAYKRYK

SEQ ID NO: 45 - protein, 120 aa, Artificial Sequence
  binding moiety for peptide tag
AMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

SEQ ID NO: 46 - protein, 751 aa, Artificial Sequence
  amino acid sequence of AAV2-588-LA-AAS-A
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLADPIVMIDNDKPITARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 47 - protein, 753 aa, Artificial Sequence
  amino acid sequence of AAV2-588-TG-AAS-GLS
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNTGDPIVMIDNDKPITGLSRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 48 - protein, 758 aa, Artificial Sequence
  amino acid sequence of AAV2-588-LA-tev-SpyTag-A
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAENLYFQGAHIVMVDAYKPTKARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 49 - protein, 760 aa, Artificial Sequence
  amino acid sequence of AAV2-588-TG-tev-SpyTag- GLS
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNTGENLYFQGAHIVMVDAYKPTKGLSRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

(19) United States
(12) Patent Application Publication
(10) Pub. No.: US 2022/0348613 A1
(43) Pub. Date: Nov. 3, 2022

(54) ADENO-ASSOCIATED VIRUSES AND METHODS AND MATERIALS FOR MAKING AND USING ADENO-ASSOCIATED VIRUSES

(71) Applicants: University of Pittsburgh - of the Commonwealath System of Higher Ecucation, Pittsburgh, PA (US); The Regents of the University of California, Oakland, CA (US)

(72) Inventors: Leah Byrne, Pittsburgh, PA (US); Bilge Esin Ozturk, Pittsburgh, PA (US); Timothy Day, Pittsburgh, PA (US)

(21) Appl. No.: 17/638,451

(22) PCT Filed: Aug. 28, 2020
(86) PCT No.: PCT/US20/48592

§ 371 Date: Feb. 25, 2022

Related U.S. Application Data
(60) Provisional application No. 62/892,959, filed on Aug. 28, 2019.

(51) Int. Cl. C07K 14/005; A61K 38/16

(52) U.S. Cl. CPC C07K 14/005; A61K 38/162; C07K 2319/50

(57) ABSTRACT

This document provides AAVs and methods and materials for making and using AAVs. For example, AAVs containing a capsid polypeptide that includes an amino acid segment having a DPIVMIDNDKPIT sequence (or a variant thereof) are provided. This document also provides compositions containing an AAV described herein, nucleic acid molecules encoding an AAV described herein, conjugating polypeptides, nucleic acid molecules encoding a conjugating polypeptide described herein, and methods for making a composition that includes two or more different AAVs covalently linked together.

### CROSS-REFERENCE TO RELATED APPLICATION

This application claims the benefit of U.S. Patent Application Ser. No. 62/892,959, filed on Aug. 28, 2019. The disclosure of the prior application is considered part of (and is incorporated by reference in) the disclosure of this application.

### BACKGROUND

### 1. Technical Field

This document relates to adeno-associated viruses (AAVs) and methods and materials for making and using AAVs. For example, this document provides AAVs containing a modified capsid polypeptide and methods for using such AAVs to produce compositions containing two or more AAVs covalently linked together.

### 2. Background Information

Viruses, such as AAVs, are efficient modes for in vivo gene transfer, and their use in the clinic is expanding. Improved AAV designs and production techniques for making effective AAV preparations should further expand the use of AAVs in the laboratory and clinic.

### SUMMARY

This document provides AAVs and methods and materials for making and using AAVs. For example, this document provides AAVs containing a capsid polypeptide that includes an amino acid segment having a DPIVMIDNDKPIT sequence (SEQ ID NO:1) or a variant thereof. Such a capsid polypeptide can provide AAVs with the ability to form a covalent bond with a polypeptide designed to include a binding moiety that covalently binds to that amino acid segment of the capsid polypeptide. For example, a conjugating polypeptide can be designed as described herein to include a binding moiety that covalently binds to an amino acid segment having SEQ ID NO:1 (or a variant thereof) of a capsid polypeptide of an AAV provided herein and used to conjugate that AAV to, for example, another AAV to form two AAVs that are covalently attached together (see, e.g., FIG. 1).

This document also provides (a) compositions containing an AAV described herein, (b) nucleic acid molecules encoding an AAV described herein, (c) conjugating polypeptides, (d) nucleic acid molecules encoding a conjugating polypeptide described herein, and (e) methods for making a composition that includes two or more different AAVs covalently linked together.

As described herein, AAVs designed to contain a capsid polypeptide that includes an amino acid segment that contains the amino acid sequence set forth in SEQ ID NO:1 (or a variant thereof) can be used to produce high titer preparations of AAVs. For example, AAVs designed to contain a capsid polypeptide that includes an amino acid segment containing the amino acid sequence set forth in SEQ ID NO:1 (or a variant thereof) can be used to produce a preparation of functional AAVs with a titer greater than 10¹⁴ (e.g., greater than 1×10¹⁴, greater than 2×10¹⁴, greater than 3×10¹⁴, greater than 4×10¹⁴, greater than 5×10¹⁴, greater than 6×10¹⁴, greater than 7×10¹⁴, greater than 8×10¹⁴, or greater than 9×10¹⁴). In some cases, an AAV designed to contain a capsid polypeptide that includes an amino acid segment containing the amino acid sequence set forth in SEQ ID NO:1 (or a variant thereof) can be used to produce an AAV preparation with a titer that is greater than (e.g., at least 5-fold, 10-fold, 25-fold, 50-fold, 75-fold, or 100-fold greater than) an AAV preparation of a comparable AAV that lacks an amino acid segment containing the amino acid sequence set forth in SEQ ID NO:1 (or a variant thereof). For example, an AAV designed to contain a capsid polypeptide that includes an amino acid segment containing the amino acid sequence set forth in SEQ ID NO:1 (or a variant thereof) can be used to produce an AAV preparation with a titer that is greater than (e.g., at least 5-fold, 10-fold, 25-fold, 50-fold, 75-fold, or 100-fold greater than) an AAV preparation of a comparable AAV that contains an amino acid tag containing the amino acid sequence set forth in SEQ ID NO:8 or SEQ ID NO:9, instead of SEQ ID NO:1 (or a variant thereof).

Having the ability to produce AAV preparations with such high titers of functional AAVs containing a capsid polypeptide provided herein can greatly increase the efficiency of using a preparation of AAVs (e.g., modified AAVs) to produce products that include a functional AAV covalently attached to another moiety of interest such as another AAV.

In general, one aspect of this document features an AAV comprising a capsid polypeptide, wherein the capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions. The AAV can be an AAV2. The amino acid segment can comprise the amino acid sequence set forth in SEQ ID NO:1 with three amino acid substitutions. The amino acid segment can comprise the amino acid sequence set forth in SEQ ID NO:1 with two amino acid substitutions. The amino acid segment can comprise the amino acid sequence set forth in SEQ ID NO:1 with one amino acid substitutions. The amino acid segment can comprise the amino acid sequence set forth in SEQ ID NO:1. The capsid polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The capsid polypeptide can comprise the amino acid segment at an amino acid position within three amino acid residues of an amino acid residue corresponding to position 453 of a reference VP1 polypeptide having the amino acid sequence set forth in SEQ ID NO:2. The capsid polypeptide can comprise the amino acid segment at an amino acid position within two amino acid residues of an amino acid residue corresponding to position 453 of a reference VP1 polypeptide having the amino acid sequence set forth in SEQ ID NO:2. The capsid polypeptide can comprise the amino acid segment at an amino acid position within one amino acid residue of an amino acid residue corresponding to position 453 of a reference VP1 polypeptide having the amino acid sequence set forth in SEQ ID NO:2. The capsid polypeptide can comprise the amino acid segment at an amino acid position of an amino acid residue corresponding to position 453 of a reference VP1 polypeptide having the amino acid sequence set forth in SEQ ID NO:2. The capsid polypeptide can comprise the amino acid segment at an amino acid position within three amino acid residues of an amino acid residue corresponding to position 588 of a reference VP1 polypeptide having the amino acid sequence set forth in SEQ ID NO:2. The capsid polypeptide can comprise the amino acid segment at an amino acid position within two amino acid residues of an amino acid residue corresponding to position 588 of a reference VP1 polypeptide having the amino acid sequence set forth in SEQ ID NO:2. The capsid polypeptide can comprise the amino acid segment at an amino acid position within one amino acid residue of an amino acid residue corresponding to position 588 of a reference VP1 polypeptide having the amino acid sequence set forth in SEQ ID NO:2. The capsid polypeptide can comprise the amino acid segment at an amino acid position of an amino acid residue corresponding to position 588 of a reference VP1 polypeptide having the amino acid sequence set forth in SEQ ID NO:2. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, and wherein the capsid polypeptide comprises an alanine immediately following the amino acid segment. The capsid polypeptide can comprise a threonine and glycine (TG) sequence immediately preceding the amino acid segment, and wherein the capsid polypeptide comprises a glycine, leucine, and serine (GLS) sequence immediately following the amino acid segment. The capsid polypeptide can comprise a protease cleavage site. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The protease cleavage site can immediately precede the amino acid segment or precedes the amino acid segment by no more than 20 amino acid residues. The protease cleavage site can precede the amino acid segment by no more than ten amino acid residues. The protease cleavage site can immediately follow the amino acid segment or follows the amino acid segment by no more than 20 amino acid residues. The protease cleavage site can follow the amino acid segment by no more than ten amino acid residues. The capsid polypeptide can comprise an N-terminal linker immediately preceding a protease cleavage site or preceding the protease cleavage site by no more than ten amino acid residues, wherein the protease cleavage site immediately precedes the amino acid segment or precedes the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the protease cleavage site, wherein the protease cleavage site immediately precedes the amino acid segment, and wherein an alanine immediately follows the amino acid segment. The capsid polypeptide can comprise a threonine and glycine (TG) sequence immediately preceding the protease cleavage site, wherein the protease cleavage site immediately precedes the amino acid segment, and wherein a glycine, leucine, and serine (GLS) sequence immediately follows the amino acid segment. The capsid polypeptide can comprise an N-terminal linker immediately preceding the amino acid segment or preceding the amino acid segment by no more than ten amino acid residues, and wherein the capsid polypeptide comprises a protease cleavage site immediately following the amino acid segment or following the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, wherein the protease cleavage site immediately follows the amino acid segment, and wherein an alanine immediately follows the protease cleavage site. The capsid polypeptide can comprise a threonine and glycine (TG) sequence immediately preceding the amino acid segment, wherein the protease cleavage site immediately follows the amino acid segment, and wherein a glycine, leucine, and serine (GLS) sequence immediately follows the protease cleavage site. The capsid polypeptide can comprise a C-terminal linker immediately following a protease cleavage site or following the protease cleavage site by no more than ten amino acid residues, and wherein the protease cleavage site immediately follows the amino acid segment or follows the amino acid segment by no more than ten amino acid residues. The C-terminal linker can comprise (a) an alanine (A) or (b) a glycine, leucine, and serine (GLS) sequence. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25.

In another aspect, this document features a composition comprising (or consisting essentially of or consisting of) an AAV comprising a capsid polypeptide, wherein the capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions. The AAV can be an AAV2. The amino acid segment can comprise the amino acid sequence set forth in SEQ ID NO:1 with three amino acid substitutions. The amino acid segment can comprise the amino acid sequence set forth in SEQ ID NO:1 with two amino acid substitutions. The amino acid segment can comprise the amino acid sequence set forth in SEQ ID NO:1 with one amino acid substitutions. The amino acid segment can comprise the amino acid sequence set forth in SEQ ID NO:1. The capsid polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The capsid polypeptide can comprise the amino acid segment at an amino acid position within three amino acid residues of an amino acid residue corresponding to position 453 of a reference VP1 polypeptide having the amino acid sequence set forth in SEQ ID NO:2. The capsid polypeptide can comprise the amino acid segment at an amino acid position within two amino acid residues of an amino acid residue corresponding to position 453 of a reference VP1 polypeptide having the amino acid sequence set forth in SEQ ID NO:2. The capsid polypeptide can comprise the amino acid segment at an amino acid position within one amino acid residue of an amino acid residue corresponding to position 453 of a reference VP1 polypeptide having the amino acid sequence set forth in SEQ ID NO:2. The capsid polypeptide can comprise the amino acid segment at an amino acid position of an amino acid residue corresponding to position 453 of a reference VP1 polypeptide having the amino acid sequence set forth in SEQ ID NO:2. The capsid polypeptide can comprise the amino acid segment at an amino acid position within three amino acid residues of an amino acid residue corresponding to position 588 of a reference VP1 polypeptide having the amino acid sequence set forth in SEQ ID NO:2. The capsid polypeptide can comprise the amino acid segment at an amino acid position within two amino acid residues of an amino acid residue corresponding to position 588 of a reference VP1 polypeptide having the amino acid sequence set forth in SEQ ID NO:2. The capsid polypeptide can comprise the amino acid segment at an amino acid position within one amino acid residue of an amino acid residue corresponding to position 588 of a reference VP1 polypeptide having the amino acid sequence set forth in SEQ ID NO:2. The capsid polypeptide can comprise the amino acid segment at an amino acid position of an amino acid residue corresponding to position 588 of a reference VP1 polypeptide having the amino acid sequence set forth in SEQ ID NO:2. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, and wherein the capsid polypeptide comprises an alanine immediately following the amino acid segment. The capsid polypeptide can comprise a threonine and glycine (TG) sequence immediately preceding the amino acid segment, and wherein the capsid polypeptide comprises a glycine, leucine, and serine (GLS) sequence immediately following the amino acid segment. The capsid polypeptide can comprise a protease cleavage site. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The protease cleavage site can immediately precede the amino acid segment or precedes the amino acid segment by no more than 20 amino acid residues. The protease cleavage site can precede the amino acid segment by no more than ten amino acid residues. The protease cleavage site can immediately follow the amino acid segment or follows the amino acid segment by no more than 20 amino acid residues. The protease cleavage site can follow the amino acid segment by no more than ten amino acid residues. The capsid polypeptide can comprise an N-terminal linker immediately preceding a protease cleavage site or preceding the protease cleavage site by no more than ten amino acid residues, wherein the protease cleavage site immediately precedes the amino acid segment or precedes the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the protease cleavage site, wherein the protease cleavage site immediately precedes the amino acid segment, and wherein an alanine immediately follows the amino acid segment. The capsid polypeptide can comprise a threonine and glycine (TG) sequence immediately preceding the protease cleavage site, wherein the protease cleavage site immediately precedes the amino acid segment, and wherein a glycine, leucine, and serine (GLS) sequence immediately follows the amino acid segment. The capsid polypeptide can comprise an N-terminal linker immediately preceding the amino acid segment or preceding the amino acid segment by no more than ten amino acid residues, and wherein the capsid polypeptide comprises a protease cleavage site immediately following the amino acid segment or following the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, wherein the protease cleavage site immediately follows the amino acid segment, and wherein an alanine immediately follows the protease cleavage site. The capsid polypeptide can comprise a threonine and glycine (TG) sequence immediately preceding the amino acid segment, wherein the protease cleavage site immediately follows the amino acid segment, and wherein a glycine, leucine, and serine (GLS) sequence immediately follows the protease cleavage site. The capsid polypeptide can comprise a C-terminal linker immediately following a protease cleavage site or following the protease cleavage site by no more than ten amino acid residues, and wherein the protease cleavage site immediately follows the amino acid segment or follows the amino acid segment by no more than ten amino acid residues. The C-terminal linker can comprise (a) an alanine (A) or (b) a glycine, leucine, and serine (GLS) sequence. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. In some cases, the titer of the AAV of the composition can be greater than 10¹⁴.

In another aspect, this document features an isolated nucleic acid encoding an AAV comprising a capsid polypeptide, wherein the capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions. The AAV can be an AAV2. The amino acid segment can comprise the amino acid sequence set forth in SEQ ID NO:1 with three amino acid substitutions. The amino acid segment can comprise the amino acid sequence set forth in SEQ ID NO:1 with two amino acid substitutions. The amino acid segment can comprise the amino acid sequence set forth in SEQ ID NO:1 with one amino acid substitutions. The amino acid segment can comprise the amino acid sequence set forth in SEQ ID NO:1. The capsid polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The capsid polypeptide can comprise the amino acid segment at an amino acid position within three amino acid residues of an amino acid residue corresponding to position 453 of a reference VP1 polypeptide having the amino acid sequence set forth in SEQ ID NO:2. The capsid polypeptide can comprise the amino acid segment at an amino acid position within two amino acid residues of an amino acid residue corresponding to position 453 of a reference VP1 polypeptide having the amino acid sequence set forth in SEQ ID NO:2. The capsid polypeptide can comprise the amino acid segment at an amino acid position within one amino acid residue of an amino acid residue corresponding to position 453 of a reference VP1 polypeptide having the amino acid sequence set forth in SEQ ID NO:2. The capsid polypeptide can comprise the amino acid segment at an amino acid position of an amino acid residue corresponding to position 453 of a reference VP1 polypeptide having the amino acid sequence set forth in SEQ ID NO:2. The capsid polypeptide can comprise the amino acid segment at an amino acid position within three amino acid residues of an amino acid residue corresponding to position 588 of a reference VP1 polypeptide having the amino acid sequence set forth in SEQ ID NO:2. The capsid polypeptide can comprise the amino acid segment at an amino acid position within two amino acid residues of an amino acid residue corresponding to position 588 of a reference VP1 polypeptide having the amino acid sequence set forth in SEQ ID NO:2. The capsid polypeptide can comprise the amino acid segment at an amino acid position within one amino acid residue of an amino acid residue corresponding to position 588 of a reference VP1 polypeptide having the amino acid sequence set forth in SEQ ID NO:2. The capsid polypeptide can comprise the amino acid segment at an amino acid position of an amino acid residue corresponding to position 588 of a reference VP1 polypeptide having the amino acid sequence set forth in SEQ ID NO:2. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, and wherein the capsid polypeptide comprises an alanine immediately following the amino acid segment. The capsid polypeptide can comprise a threonine and glycine (TG) sequence immediately preceding the amino acid segment, and wherein the capsid polypeptide comprises a glycine, leucine, and serine (GLS) sequence immediately following the amino acid segment. The capsid polypeptide can comprise a protease cleavage site. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The protease cleavage site can immediately precede the amino acid segment or precedes the amino acid segment by no more than 20 amino acid residues. The protease cleavage site can precede the amino acid segment by no more than ten amino acid residues. The protease cleavage site can immediately follow the amino acid segment or follows the amino acid segment by no more than 20 amino acid residues. The protease cleavage site can follow the amino acid segment by no more than ten amino acid residues. The capsid polypeptide can comprise an N-terminal linker immediately preceding a protease cleavage site or preceding the protease cleavage site by no more than ten amino acid residues, wherein the protease cleavage site immediately precedes the amino acid segment or precedes the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the protease cleavage site, wherein the protease cleavage site immediately precedes the amino acid segment, and wherein an alanine immediately follows the amino acid segment. The capsid polypeptide can comprise a threonine and glycine (TG) sequence immediately preceding the protease cleavage site, wherein the protease cleavage site immediately precedes the amino acid segment, and wherein a glycine, leucine, and serine (GLS) sequence immediately follows the amino acid segment. The capsid polypeptide can comprise an N-terminal linker immediately preceding the amino acid segment or preceding the amino acid segment by no more than ten amino acid residues, and wherein the capsid polypeptide comprises a protease cleavage site immediately following the amino acid segment or following the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, wherein the protease cleavage site immediately follows the amino acid segment, and wherein an alanine immediately follows the protease cleavage site. The capsid polypeptide can comprise a threonine and glycine (TG) sequence immediately preceding the amino acid segment, wherein the protease cleavage site immediately follows the amino acid segment, and wherein a glycine, leucine, and serine (GLS) sequence immediately follows the protease cleavage site. The capsid polypeptide can comprise a C-terminal linker immediately following a protease cleavage site or following the protease cleavage site by no more than ten amino acid residues, and wherein the protease cleavage site immediately follows the amino acid segment or follows the amino acid segment by no more than ten amino acid residues. The C-terminal linker can comprise (a) an alanine (A) or (b) a glycine, leucine, and serine (GLS) sequence. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25.

In another aspect, this document features a composition comprising (or consisting essentially of or consisting of) a first AAV and a second AAV genetically different from the first AAV, wherein the first AAV is an AAV comprising a capsid polypeptide, wherein the capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions. The first AAV can be an AAV2. The amino acid segment can comprise the amino acid sequence set forth in SEQ ID NO:1 with three amino acid substitutions. The amino acid segment can comprise the amino acid sequence set forth in SEQ ID NO:1 with two amino acid substitutions. The amino acid segment can comprise the amino acid sequence set forth in SEQ ID NO:1 with one amino acid substitutions. The amino acid segment can comprise the amino acid sequence set forth in SEQ ID NO:1. The capsid polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The capsid polypeptide can comprise the amino acid segment at an amino acid position within three amino acid residues of an amino acid residue corresponding to position 453 of a reference VP1 polypeptide having the amino acid sequence set forth in SEQ ID NO:2. The capsid polypeptide can comprise the amino acid segment at an amino acid position within two amino acid residues of an amino acid residue corresponding to position 453 of a reference VP1 polypeptide having the amino acid sequence set forth in SEQ ID NO:2. The capsid polypeptide can comprise the amino acid segment at an amino acid position within one amino acid residue of an amino acid residue corresponding to position 453 of a reference VP1 polypeptide having the amino acid sequence set forth in SEQ ID NO:2. The capsid polypeptide can comprise the amino acid segment at an amino acid position of an amino acid residue corresponding to position 453 of a reference VP1 polypeptide having the amino acid sequence set forth in SEQ ID NO:2. The capsid polypeptide can comprise the amino acid segment at an amino acid position within three amino acid residues of an amino acid residue corresponding to position 588 of a reference VP1 polypeptide having the amino acid sequence set forth in SEQ ID NO:2. The capsid polypeptide can comprise the amino acid segment at an amino acid position within two amino acid residues of an amino acid residue corresponding to position 588 of a reference VP1 polypeptide having the amino acid sequence set forth in SEQ ID NO:2. The capsid polypeptide can comprise the amino acid segment at an amino acid position within one amino acid residue of an amino acid residue corresponding to position 588 of a reference VP1 polypeptide having the amino acid sequence set forth in SEQ ID NO:2. The capsid polypeptide can comprise the amino acid segment at an amino acid position of an amino acid residue corresponding to position 588 of a reference VP1 polypeptide having the amino acid sequence set forth in SEQ ID NO:2. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, and wherein the capsid polypeptide comprises an alanine immediately following the amino acid segment. The capsid polypeptide can comprise a threonine and glycine (TG) sequence immediately preceding the amino acid segment, and wherein the capsid polypeptide comprises a glycine, leucine, and serine (GLS) sequence immediately following the amino acid segment. The capsid polypeptide can comprise a protease cleavage site. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The protease cleavage site can immediately precede the amino acid segment or precedes the amino acid segment by no more than 20 amino acid residues. The protease cleavage site can precede the amino acid segment by no more than ten amino acid residues. The protease cleavage site can immediately follow the amino acid segment or follows the amino acid segment by no more than 20 amino acid residues. The protease cleavage site can follow the amino acid segment by no more than ten amino acid residues. The capsid polypeptide can comprise an N-terminal linker immediately preceding a protease cleavage site or preceding the protease cleavage site by no more than ten amino acid residues, wherein the protease cleavage site immediately precedes the amino acid segment or precedes the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the protease cleavage site, wherein the protease cleavage site immediately precedes the amino acid segment, and wherein an alanine immediately follows the amino acid segment. The capsid polypeptide can comprise a threonine and glycine (TG) sequence immediately preceding the protease cleavage site, wherein the protease cleavage site immediately precedes the amino acid segment, and wherein a glycine, leucine, and serine (GLS) sequence immediately follows the amino acid segment. The capsid polypeptide can comprise an N-terminal linker immediately preceding the amino acid segment or preceding the amino acid segment by no more than ten amino acid residues, and wherein the capsid polypeptide comprises a protease cleavage site immediately following the amino acid segment or following the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, wherein the protease cleavage site immediately follows the amino acid segment, and wherein an alanine immediately follows the protease cleavage site. The capsid polypeptide can comprise a threonine and glycine (TG) sequence immediately preceding the amino acid segment, wherein the protease cleavage site immediately follows the amino acid segment, and wherein a glycine, leucine, and serine (GLS) sequence immediately follows the protease cleavage site. The capsid polypeptide can comprise a C-terminal linker immediately following a protease cleavage site or following the protease cleavage site by no more than ten amino acid residues, and wherein the protease cleavage site immediately follows the amino acid segment or follows the amino acid segment by no more than ten amino acid residues. The C-terminal linker can comprise (a) an alanine (A) or (b) a glycine, leucine, and serine (GLS) sequence. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25.

In some cases, first and second AAVs can be covalently attached to each other. The first AAV can be covalently attached to the second AAV via at least one covalent bond of the amino acid segment of the first AAV. The capsid polypeptides of the first and second AAVs can be identical. The capsid polypeptides of the first and second AAVs can be different. The capsid polypeptides of the second AAV can lack the amino acid segment. The second AAV can comprise a capsid polypeptide comprising an amino acid tag comprising: (a) the amino acid sequence set forth in SEQ ID NO:1 or the amino acid sequence set forth in SEQ ID NO:1 with one, two, or three amino acid substitutions, (b) the amino acid sequence set forth in SEQ ID NO:8 or the amino acid sequence set forth in SEQ ID NO:8 with one, two, or three amino acid substitutions, (c) the amino acid sequence set forth in SEQ ID NO:9 or the amino acid sequence set forth in SEQ ID NO:9 with one, two, or three amino acid substitutions, or (d) the amino acid sequence set forth in SEQ ID NO:44 or the amino acid sequence set forth in SEQ ID NO:44 with one, two, or three amino acid substitutions. The capsid polypeptide of the second AAV can comprise the amino acid tag at an amino acid position within three amino acid residues of an amino acid residue corresponding to position 453 or position 588 of a reference VP1 polypeptide having the amino acid sequence set forth in SEQ ID NO:2. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, and wherein the capsid polypeptide of the second AAV comprises an alanine immediately following the amino acid tag. The capsid polypeptide of the second AAV can comprise a threonine and glycine (TG) sequence immediately preceding the amino acid tag, and wherein the capsid polypeptide of the second AAV comprises a glycine, leucine, and serine (GLS) sequence immediately following the amino acid tag. The capsid polypeptide of the second AAV can comprise a protease cleavage site. The capsid polypeptide of the second AAV can comprise a protease cleavage site. The protease cleavage site of the second AAV can immediately precede the amino acid tag or precedes the amino acid tag by no more than 20 amino acid residues. The protease cleavage site of the second AAV can precede the amino acid tag by no more than ten amino acid residues. The protease cleavage site of the second AAV can immediately follow the amino acid tag or follows the amino acid tag by no more than 20 amino acid residues. The protease cleavage site of the second AAV can follow the amino acid tag by no more than ten amino acid residues. The capsid polypeptide of the second AAV can comprise an N-terminal linker immediately preceding the protease cleavage site or preceding the protease cleavage site by no more than ten amino acid residues, wherein the protease cleavage site immediately precedes the amino acid tag or precedes the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the protease cleavage site, wherein the protease cleavage site immediately precedes the amino acid tag, and wherein an alanine immediately follows the amino acid tag. The capsid polypeptide of the second AAV can comprise a threonine and glycine (TG) sequence immediately preceding the protease cleavage site, wherein the protease cleavage site immediately precedes the amino acid tag, and wherein a glycine, leucine, and serine (GLS) sequence immediately follows the amino acid tag. The capsid polypeptide of the second AAV can comprise an N-terminal linker immediately preceding the amino acid tag or preceding the amino acid tag by no more than ten amino acid residues, wherein the protease cleavage site immediately follows the amino acid tag or follows the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, wherein the protease cleavage site immediately follows the amino acid tag, and wherein an alanine immediately follows the protease cleavage site. The capsid polypeptide of the second AAV can comprise a threonine and glycine (TG) sequence immediately preceding the amino acid tag, wherein the protease cleavage site immediately follows the amino acid tag, and wherein a glycine, leucine, and serine (GLS) sequence immediately follows the protease cleavage site. The capsid polypeptide of the second AAV can comprise a C-terminal linker immediately following the protease cleavage site or following the protease cleavage site by no more than ten amino acid residues, and wherein the protease cleavage site immediately follows the amino acid tag or follows the amino acid tag by no more than ten amino acid residues. The C-terminal linker can comprise (a) an alanine (A) or (b) a glycine, leucine, and serine (GLS) sequence. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The second AAV can be an AAV2. The composition can comprise a conjugating polypeptide having the ability to bind covalently to the amino acid segment of the first AAV. The conjugating polypeptide can comprise a first binding motif. The first binding motif can comprise (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The first binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:11. The first binding motif can comprise the amino acid sequence set forth in SEQ ID NO:10, SEQ ID NO:26, or SEQ ID NO:27. The first binding motif can comprise (i) the amino acid sequence set forth in SEQ ID NO:12 or (ii) the amino acid sequence set forth in SEQ ID NO:12 with 30 or less amino acid additions, deletions, or substitutions with the proviso that the amino acid located at a position corresponding to position 31 of SEQ ID NO:12 remains a lysine, with the proviso that the amino acid located at a position corresponding to position 77 of SEQ ID NO:12 remains a glutamic acid, and with the proviso that the amino acid located at a position corresponding to position 117 of SEQ ID NO:12 remains an aspartic acid. The first binding motif can comprise the amino acid sequence set forth in SEQ ID NO:12 with 25 or less amino acid additions, deletions, or substitutions with the proviso that the amino acid located at a position corresponding to position 31 of SEQ ID NO:12 remains a lysine, with the proviso that the amino acid located at a position corresponding to position 77 of SEQ ID NO:12 remains a glutamic acid, and with the proviso that the amino acid located at a position corresponding to position 117 of SEQ ID NO:12 remains an aspartic acid. The first binding motif can comprise the amino acid sequence set forth in SEQ ID NO:12 with 20 or less amino acid additions, deletions, or substitutions with the proviso that the amino acid located at a position corresponding to position 31 of SEQ ID NO:12 remains a lysine, with the proviso that the amino acid located at a position corresponding to position 77 of SEQ ID NO:12 remains a glutamic acid, and with the proviso that the amino acid located at a position corresponding to position 117 of SEQ ID NO:12 remains an aspartic acid. The first binding motif can comprise the amino acid sequence set forth in SEQ ID NO:13. The first binding motif can comprise the amino acid sequence set forth in SEQ ID NO:12. The first binding motif can comprise the amino acid sequence set forth in any one of: SEQ ID NO:14, SEQ ID NO:15, SEQ ID NO:16, and SEQ ID NO:17. The composition can comprise the conjugating polypeptide covalently attached to the amino acid segment of the first AAV. The conjugating polypeptide can comprise the ability to bind covalently to the amino acid tag of the second AAV. The composition can comprise the conjugating polypeptide covalently attached to the amino acid segment of the first AAV and to the amino acid tag of the second AAV.

In some cases, the conjugating polypeptide can comprise two of the first binding motifs as follows in this paragraph. For example, the first binding motif can comprise (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The first binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:11. The first binding motif can comprise the amino acid sequence set forth in SEQ ID NO:10, SEQ ID NO:26, or SEQ ID NO:27. The first binding motif can comprise (i) the amino acid sequence set forth in SEQ ID NO:12 or (ii) the amino acid sequence set forth in SEQ ID NO:12 with 30 or less amino acid additions, deletions, or substitutions with the proviso that the amino acid located at a position corresponding to position 31 of SEQ ID NO:12 remains a lysine, with the proviso that the amino acid located at a position corresponding to position 77 of SEQ ID NO:12 remains a glutamic acid, and with the proviso that the amino acid located at a position corresponding to position 117 of SEQ ID NO:12 remains an aspartic acid. The first binding motif can comprise the amino acid sequence set forth in SEQ ID NO:12 with 25 or less amino acid additions, deletions, or substitutions with the proviso that the amino acid located at a position corresponding to position 31 of SEQ ID NO:12 remains a lysine, with the proviso that the amino acid located at a position corresponding to position 77 of SEQ ID NO:12 remains a glutamic acid, and with the proviso that the amino acid located at a position corresponding to position 117 of SEQ ID NO:12 remains an aspartic acid. The first binding motif can comprise the amino acid sequence set forth in SEQ ID NO:12 with 20 or less amino acid additions, deletions, or substitutions with the proviso that the amino acid located at a position corresponding to position 31 of SEQ ID NO:12 remains a lysine, with the proviso that the amino acid located at a position corresponding to position 77 of SEQ ID NO:12 remains a glutamic acid, and with the proviso that the amino acid located at a position corresponding to position 117 of SEQ ID NO:12 remains an aspartic acid. The first binding motif can comprise the amino acid sequence set forth in SEQ ID NO:13. The first binding motif can comprise the amino acid sequence set forth in SEQ ID NO:12. The first binding motif can comprise the amino acid sequence set forth in any one of: SEQ ID NO:14, SEQ ID NO:15, SEQ ID NO:16, and SEQ ID NO:17. The amino acid sequence of each of the two can be identical. The amino acid sequence of each of the two can be different. The two of the first binding motifs can be separated by a linker. The linker can comprise the amino acid sequence set forth in SEQ ID NO:41, SEQ ID NO:42, or SEQ ID NO:43.

In some cases, the conjugating polypeptide can comprise a second binding motif. The second binding motif can comprise the ability to bind covalently to the amino acid tag of the second AAV. The second binding motif can comprise (a) the amino acid sequence set forth in SEQ ID NO:18 or the amino acid sequence set forth in SEQ ID NO:18 with 25 or less amino acid additions, deletions, or substitutions, (b) the amino acid sequence set forth in SEQ ID NO:19 or the amino acid sequence set forth in SEQ ID NO:19 with 25 or less amino acid additions, deletions, or substitutions, or (c) the amino acid sequence set forth in SEQ ID NO:45 or the amino acid sequence set forth in SEQ ID NO:45 with 25 or less amino acid additions, deletions, or substitutions. The first binding motif and the second binding motif can be separated by a linker. The linker can comprise the amino acid sequence set forth in SEQ ID NO:41, SEQ ID NO:42, or SEQ ID NO:43. The conjugating polypeptide can comprise SEQ ID NO:41.

In another aspect, this document features a conjugating polypeptide comprising (or consisting essentially of or consisting of) (a) a first binding motif and (b) a second binding motif, wherein the first binding motif comprises (i) the amino acid sequence set forth in SEQ ID NO:10 (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The first binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:11. The first binding motif can comprise the amino acid sequence set forth in SEQ ID NO:10, SEQ ID NO:26, or SEQ ID NO:27. The second binding motif can comprise (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The second binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:11. The second binding motif can comprise the amino acid sequence set forth in SEQ ID NO:10, SEQ ID NO:26, SEQ ID NO:27. The second binding motif can comprise (a) the amino acid sequence set forth in SEQ ID NO:18 or the amino acid sequence set forth in SEQ ID NO:18 with 25 or less amino acid additions, deletions, or substitutions, (b) the amino acid sequence set forth in SEQ ID NO:19 or the amino acid sequence set forth in SEQ ID NO:19 with 25 or less amino acid additions, deletions, or substitutions, or (c) the amino acid sequence set forth in SEQ ID NO:45 or the amino acid sequence set forth in SEQ ID NO:45 with 25 or less amino acid additions, deletions, or substitutions. The first binding motif and the second binding motif can be separated by a linker. The linker can comprise the amino acid sequence set forth in SEQ ID NO:41, SEQ ID NO:42, or SEQ ID NO:43. The conjugating polypeptide can comprise SEQ ID NO:41.

In another aspect, this document features an isolated nucleic acid comprising (or consisting essentially of or consisting of) a nucleic acid sequence encoding a conjugating polypeptide comprising (or consisting essentially of or consisting of) (a) a first binding motif and (b) a second binding motif, wherein the first binding motif comprises (i) the amino acid sequence set forth in SEQ ID NO:10 (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The first binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:11. The first binding motif can comprise the amino acid sequence set forth in SEQ ID NO:10, SEQ ID NO:26, or SEQ ID NO:27. The second binding motif can comprise (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The second binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:11. The second binding motif can comprise the amino acid sequence set forth in SEQ ID NO:10, SEQ ID NO:26, SEQ ID NO:27. The second binding motif can comprise (a) the amino acid sequence set forth in SEQ ID NO:18 or the amino acid sequence set forth in SEQ ID NO:18 with 25 or less amino acid additions, deletions, or substitutions, (b) the amino acid sequence set forth in SEQ ID NO:19 or the amino acid sequence set forth in SEQ ID NO:19 with 25 or less amino acid additions, deletions, or substitutions, or (c) the amino acid sequence set forth in SEQ ID NO:45 or the amino acid sequence set forth in SEQ ID NO:45 with 25 or less amino acid additions, deletions, or substitutions. The first binding motif and the second binding motif can be separated by a linker. The linker can comprise the amino acid sequence set forth in SEQ ID NO:41, SEQ ID NO:42, or SEQ ID NO:43. The conjugating polypeptide can comprise SEQ ID NO:41.

In another aspect, this document features a method for producing a composition comprising (or consisting essentially of or consisting of) a first AAV covalently linked to a second AAV, wherein the first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, wherein the second AAV comprises a capsid polypeptide comprising an amino acid tag. The method comprises (or consists essentially of or consists of) (a) contacting the first AAV with a conjugating polypeptide to form a first AAV-conjugating polypeptide complex, wherein the conjugating polypeptide comprises a first binding motif having the ability to bind covalently to the amino acid segment of the first AAV and a second binding motif having the ability to bind covalently to the amino acid tag of the second AAV, and (b) contacting the first AAV-conjugating polypeptide complex with the second AAV for form a first AAV-conjugating polypeptide-second AAV complex, thereby producing the composition comprising the first AAV covalently linked to the second AAV. The contacting step with the first AAV can comprise using a solution comprising a titer of the AAV that is greater than 10¹⁴.

In some cases, the first AAV can be an AAV comprising a capsid polypeptide, wherein the capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions. The AAV can be an AAV2. The amino acid segment can comprise the amino acid sequence set forth in SEQ ID NO:1 with three amino acid substitutions. The amino acid segment can comprise the amino acid sequence set forth in SEQ ID NO:1 with two amino acid substitutions. The amino acid segment can comprise the amino acid sequence set forth in SEQ ID NO:1 with one amino acid substitutions. The amino acid segment can comprise the amino acid sequence set forth in SEQ ID NO:1. The capsid polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The capsid polypeptide can comprise the amino acid segment at an amino acid position within three amino acid residues of an amino acid residue corresponding to position 453 of a reference VP1 polypeptide having the amino acid sequence set forth in SEQ ID NO:2. The capsid polypeptide can comprise the amino acid segment at an amino acid position within two amino acid residues of an amino acid residue corresponding to position 453 of a reference VP1 polypeptide having the amino acid sequence set forth in SEQ ID NO:2. The capsid polypeptide can comprise the amino acid segment at an amino acid position within one amino acid residue of an amino acid residue corresponding to position 453 of a reference VP1 polypeptide having the amino acid sequence set forth in SEQ ID NO:2. The capsid polypeptide can comprise the amino acid segment at an amino acid position of an amino acid residue corresponding to position 453 of a reference VP1 polypeptide having the amino acid sequence set forth in SEQ ID NO:2. The capsid polypeptide can comprise the amino acid segment at an amino acid position within three amino acid residues of an amino acid residue corresponding to position 588 of a reference VP1 polypeptide having the amino acid sequence set forth in SEQ ID NO:2. The capsid polypeptide can comprise the amino acid segment at an amino acid position within two amino acid residues of an amino acid residue corresponding to position 588 of a reference VP1 polypeptide having the amino acid sequence set forth in SEQ ID NO:2. The capsid polypeptide can comprise the amino acid segment at an amino acid position within one amino acid residue of an amino acid residue corresponding to position 588 of a reference VP1 polypeptide having the amino acid sequence set forth in SEQ ID NO:2. The capsid polypeptide can comprise the amino acid segment at an amino acid position of an amino acid residue corresponding to position 588 of a reference VP1 polypeptide having the amino acid sequence set forth in SEQ ID NO:2. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, and wherein the capsid polypeptide comprises an alanine immediately following the amino acid segment. The capsid polypeptide can comprise a threonine and glycine (TG) sequence immediately preceding the amino acid segment, and wherein the capsid polypeptide comprises a glycine, leucine, and serine (GLS) sequence immediately following the amino acid segment. The capsid polypeptide can comprise a protease cleavage site. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The protease cleavage site can immediately precede the amino acid segment or precedes the amino acid segment by no more than 20 amino acid residues. The protease cleavage site can precede the amino acid segment by no more than ten amino acid residues. The protease cleavage site can immediately follow the amino acid segment or follows the amino acid segment by no more than 20 amino acid residues. The protease cleavage site can follow the amino acid segment by no more than ten amino acid residues. The capsid polypeptide can comprise an N-terminal linker immediately preceding a protease cleavage site or preceding the protease cleavage site by no more than ten amino acid residues, wherein the protease cleavage site immediately precedes the amino acid segment or precedes the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the protease cleavage site, wherein the protease cleavage site immediately precedes the amino acid segment, and wherein an alanine immediately follows the amino acid segment. The capsid polypeptide can comprise a threonine and glycine (TG) sequence immediately preceding the protease cleavage site, wherein the protease cleavage site immediately precedes the amino acid segment, and wherein a glycine, leucine, and serine (GLS) sequence immediately follows the amino acid segment. The capsid polypeptide can comprise an N-terminal linker immediately preceding the amino acid segment or preceding the amino acid segment by no more than ten amino acid residues, and wherein the capsid polypeptide comprises a protease cleavage site immediately following the amino acid segment or following the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, wherein the protease cleavage site immediately follows the amino acid segment, and wherein an alanine immediately follows the protease cleavage site. The capsid polypeptide can comprise a threonine and glycine (TG) sequence immediately preceding the amino acid segment, wherein the protease cleavage site immediately follows the amino acid segment, and wherein a glycine, leucine, and serine (GLS) sequence immediately follows the protease cleavage site. The capsid polypeptide can comprise a C-terminal linker immediately following a protease cleavage site or following the protease cleavage site by no more than ten amino acid residues, and wherein the protease cleavage site immediately follows the amino acid segment or follows the amino acid segment by no more than ten amino acid residues. The C-terminal linker can comprise (a) an alanine (A) or (b) a glycine, leucine, and serine (GLS) sequence. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25.

In some cases, the second AAV can be as described in this paragraph. For example, the capsid polypeptides of the second AAV can lack the amino acid segment. The second AAV can comprise a capsid polypeptide comprising an amino acid tag comprising: (a) the amino acid sequence set forth in SEQ ID NO:1 or the amino acid sequence set forth in SEQ ID NO:1 with one, two, or three amino acid substitutions, (b) the amino acid sequence set forth in SEQ ID NO:8 or the amino acid sequence set forth in SEQ ID NO:8 with one, two, or three amino acid substitutions, (c) the amino acid sequence set forth in SEQ ID NO:9 or the amino acid sequence set forth in SEQ ID NO:9 with one, two, or three amino acid substitutions, or (d) the amino acid sequence set forth in SEQ ID NO:44 or the amino acid sequence set forth in SEQ ID NO:44 with one, two, or three amino acid substitutions. The capsid polypeptide of the second AAV can comprise the amino acid tag at an amino acid position within three amino acid residues of an amino acid residue corresponding to position 453 or position 588 of a reference VP1 polypeptide having the amino acid sequence set forth in SEQ ID NO:2. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, and wherein the capsid polypeptide of the second AAV comprises an alanine immediately following the amino acid tag. The capsid polypeptide of the second AAV can comprise a threonine and glycine (TG) sequence immediately preceding the amino acid tag, and wherein the capsid polypeptide of the second AAV comprises a glycine, leucine, and serine (GLS) sequence immediately following the amino acid tag. The capsid polypeptide of the second AAV can comprise a protease cleavage site. The capsid polypeptide of the second AAV can comprise a protease cleavage site. The protease cleavage site of the second AAV can immediately precede the amino acid tag or precedes the amino acid tag by no more than 20 amino acid residues. The protease cleavage site of the second AAV can precede the amino acid tag by no more than ten amino acid residues. The protease cleavage site of the second AAV can immediately follow the amino acid tag or follows the amino acid tag by no more than 20 amino acid residues. The protease cleavage site of the second AAV can follow the amino acid tag by no more than ten amino acid residues. The capsid polypeptide of the second AAV can comprise an N-terminal linker immediately preceding the protease cleavage site or preceding the protease cleavage site by no more than ten amino acid residues, wherein the protease cleavage site immediately precedes the amino acid tag or precedes the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the protease cleavage site, wherein the protease cleavage site immediately precedes the amino acid tag, and wherein an alanine immediately follows the amino acid tag. The capsid polypeptide of the second AAV can comprise a threonine and glycine (TG) sequence immediately preceding the protease cleavage site, wherein the protease cleavage site immediately precedes the amino acid tag, and wherein a glycine, leucine, and serine (GLS) sequence immediately follows the amino acid tag. The capsid polypeptide of the second AAV can comprise an N-terminal linker immediately preceding the amino acid tag or preceding the amino acid tag by no more than ten amino acid residues, wherein the protease cleavage site immediately follows the amino acid tag or follows the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, wherein the protease cleavage site immediately follows the amino acid tag, and wherein an alanine immediately follows the protease cleavage site. The capsid polypeptide of the second AAV can comprise a threonine and glycine (TG) sequence immediately preceding the amino acid tag, wherein the protease cleavage site immediately follows the amino acid tag, and wherein a glycine, leucine, and serine (GLS) sequence immediately follows the protease cleavage site. The capsid polypeptide of the second AAV can comprise a C-terminal linker immediately following the protease cleavage site or following the protease cleavage site by no more than ten amino acid residues, and wherein the protease cleavage site immediately follows the amino acid tag or follows the amino acid tag by no more than ten amino acid residues. The C-terminal linker can comprise (a) an alanine (A) or (b) a glycine, leucine, and serine (GLS) sequence. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The second AAV can be an AAV2.

In some cases, the conjugating polypeptide can be a conjugating polypeptide comprising (or consisting essentially of or consisting of) (a) a first binding motif and (b) a second binding motif, wherein the first binding motif comprises (i) the amino acid sequence set forth in SEQ ID NO:10 (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The first binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:11. The first binding motif can comprise the amino acid sequence set forth in SEQ ID NO:10, SEQ ID NO:26, or SEQ ID NO:27. The second binding motif can comprise (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The second binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:11. The second binding motif can comprise the amino acid sequence set forth in SEQ ID NO:10, SEQ ID NO:26, SEQ ID NO:27. The second binding motif can comprise (a) the amino acid sequence set forth in SEQ ID NO:18 or the amino acid sequence set forth in SEQ ID NO:18 with 25 or less amino acid additions, deletions, or substitutions, (b) the amino acid sequence set forth in SEQ ID NO:19 or the amino acid sequence set forth in SEQ ID NO:19 with 25 or less amino acid additions, deletions, or substitutions, or (c) the amino acid sequence set forth in SEQ ID NO:45 or the amino acid sequence set forth in SEQ ID NO:45 with 25 or less amino acid additions, deletions, or substitutions. The first binding motif and the second binding motif can be separated by a linker. The linker can comprise the amino acid sequence set forth in SEQ ID NO:41, SEQ ID NO:42, or SEQ ID NO:43. The conjugating polypeptide can comprise SEQ ID NO:41.

In some cases, the method can comprise performing a purification procedure after the step (a) to form a composition enriched for the first AAV-conjugating polypeptide complex, wherein the composition enriched for the first AAV-conjugating polypeptide complex is used in the step (b). The method can comprise performing a purification procedure after the step (b) to form a composition enriched for the first AAV-conjugating polypeptide-second AAV complex.

In another aspect, this document features a method for producing a composition comprising a first AAV covalently linked to a second AAV, wherein the first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, wherein the second AAV comprises a capsid polypeptide comprising an amino acid tag. The method comprises (or consists essentially of or consists of) (a) contacting the second AAV with a conjugating polypeptide to form a second AAV-conjugating polypeptide complex, wherein the conjugating polypeptide comprises a first binding motif having the ability to bind covalently to the amino acid segment of the first AAV and a second binding motif having the ability to bind covalently to the amino acid tag of the second AAV, and (b) contacting the second AAV-conjugating polypeptide complex with the first AAV for form a second AAV-conjugating polypeptide-first AAV complex, thereby producing the composition comprising the first AAV covalently linked to the second AAV. The contacting step with the first AAV can comprise using a solution comprising a titer of the AAV that is greater than 10¹⁴.

In some cases, the first AAV can be an AAV comprising a capsid polypeptide, wherein the capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions. The AAV can be an AAV2. The amino acid segment can comprise the amino acid sequence set forth in SEQ ID NO:1 with three amino acid substitutions. The amino acid segment can comprise the amino acid sequence set forth in SEQ ID NO:1 with two amino acid substitutions. The amino acid segment can comprise the amino acid sequence set forth in SEQ ID NO:1 with one amino acid substitutions. The amino acid segment can comprise the amino acid sequence set forth in SEQ ID NO:1. The capsid polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The capsid polypeptide can comprise the amino acid segment at an amino acid position within three amino acid residues of an amino acid residue corresponding to position 453 of a reference VP1 polypeptide having the amino acid sequence set forth in SEQ ID NO:2. The capsid polypeptide can comprise the amino acid segment at an amino acid position within two amino acid residues of an amino acid residue corresponding to position 453 of a reference VP1 polypeptide having the amino acid sequence set forth in SEQ ID NO:2. The capsid polypeptide can comprise the amino acid segment at an amino acid position within one amino acid residue of an amino acid residue corresponding to position 453 of a reference VP1 polypeptide having the amino acid sequence set forth in SEQ ID NO:2. The capsid polypeptide can comprise the amino acid segment at an amino acid position of an amino acid residue corresponding to position 453 of a reference VP1 polypeptide having the amino acid sequence set forth in SEQ ID NO:2. The capsid polypeptide can comprise the amino acid segment at an amino acid position within three amino acid residues of an amino acid residue corresponding to position 588 of a reference VP1 polypeptide having the amino acid sequence set forth in SEQ ID NO:2. The capsid polypeptide can comprise the amino acid segment at an amino acid position within two amino acid residues of an amino acid residue corresponding to position 588 of a reference VP1 polypeptide having the amino acid sequence set forth in SEQ ID NO:2. The capsid polypeptide can comprise the amino acid segment at an amino acid position within one amino acid residue of an amino acid residue corresponding to position 588 of a reference VP1 polypeptide having the amino acid sequence set forth in SEQ ID NO:2. The capsid polypeptide can comprise the amino acid segment at an amino acid position of an amino acid residue corresponding to position 588 of a reference VP1 polypeptide having the amino acid sequence set forth in SEQ ID NO:2. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, and wherein the capsid polypeptide comprises an alanine immediately following the amino acid segment. The capsid polypeptide can comprise a threonine and glycine (TG) sequence immediately preceding the amino acid segment, and wherein the capsid polypeptide comprises a glycine, leucine, and serine (GLS) sequence immediately following the amino acid segment. The capsid polypeptide can comprise a protease cleavage site. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The protease cleavage site can immediately precede the amino acid segment or precedes the amino acid segment by no more than 20 amino acid residues. The protease cleavage site can precede the amino acid segment by no more than ten amino acid residues. The protease cleavage site can immediately follow the amino acid segment or follows the amino acid segment by no more than 20 amino acid residues. The protease cleavage site can follow the amino acid segment by no more than ten amino acid residues. The capsid polypeptide can comprise an N-terminal linker immediately preceding a protease cleavage site or preceding the protease cleavage site by no more than ten amino acid residues, wherein the protease cleavage site immediately precedes the amino acid segment or precedes the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the protease cleavage site, wherein the protease cleavage site immediately precedes the amino acid segment, and wherein an alanine immediately follows the amino acid segment. The capsid polypeptide can comprise a threonine and glycine (TG) sequence immediately preceding the protease cleavage site, wherein the protease cleavage site immediately precedes the amino acid segment, and wherein a glycine, leucine, and serine (GLS) sequence immediately follows the amino acid segment. The capsid polypeptide can comprise an N-terminal linker immediately preceding the amino acid segment or preceding the amino acid segment by no more than ten amino acid residues, and wherein the capsid polypeptide comprises a protease cleavage site immediately following the amino acid segment or following the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, wherein the protease cleavage site immediately follows the amino acid segment, and wherein an alanine immediately follows the protease cleavage site. The capsid polypeptide can comprise a threonine and glycine (TG) sequence immediately preceding the amino acid segment, wherein the protease cleavage site immediately follows the amino acid segment, and wherein a glycine, leucine, and serine (GLS) sequence immediately follows the protease cleavage site. The capsid polypeptide can comprise a C-terminal linker immediately following a protease cleavage site or following the protease cleavage site by no more than ten amino acid residues, and wherein the protease cleavage site immediately follows the amino acid segment or follows the amino acid segment by no more than ten amino acid residues. The C-terminal linker can comprise (a) an alanine (A) or (b) a glycine, leucine, and serine (GLS) sequence. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25.

In some cases, the second AAV can be as described in this paragraph. For example, the capsid polypeptides of the second AAV can lack the amino acid segment. The second AAV can comprise a capsid polypeptide comprising an amino acid tag comprising: (a) the amino acid sequence set forth in SEQ ID NO:1 or the amino acid sequence set forth in SEQ ID NO:1 with one, two, or three amino acid substitutions, (b) the amino acid sequence set forth in SEQ ID NO:8 or the amino acid sequence set forth in SEQ ID NO:8 with one, two, or three amino acid substitutions, (c) the amino acid sequence set forth in SEQ ID NO:9 or the amino acid sequence set forth in SEQ ID NO:9 with one, two, or three amino acid substitutions, or (d) the amino acid sequence set forth in SEQ ID NO:44 or the amino acid sequence set forth in SEQ ID NO:44 with one, two, or three amino acid substitutions. The capsid polypeptide of the second AAV can comprise the amino acid tag at an amino acid position within three amino acid residues of an amino acid residue corresponding to position 453 or position 588 of a reference VP1 polypeptide having the amino acid sequence set forth in SEQ ID NO:2. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, and wherein the capsid polypeptide of the second AAV comprises an alanine immediately following the amino acid tag. The capsid polypeptide of the second AAV can comprise a threonine and glycine (TG) sequence immediately preceding the amino acid tag, and wherein the capsid polypeptide of the second AAV comprises a glycine, leucine, and serine (GLS) sequence immediately following the amino acid tag. The capsid polypeptide of the second AAV can comprise a protease cleavage site. The capsid polypeptide of the second AAV can comprise a protease cleavage site. The protease cleavage site of the second AAV can immediately precede the amino acid tag or precedes the amino acid tag by no more than 20 amino acid residues. The protease cleavage site of the second AAV can precede the amino acid tag by no more than ten amino acid residues. The protease cleavage site of the second AAV can immediately follow the amino acid tag or follows the amino acid tag by no more than 20 amino acid residues. The protease cleavage site of the second AAV can follow the amino acid tag by no more than ten amino acid residues. The capsid polypeptide of the second AAV can comprise an N-terminal linker immediately preceding the protease cleavage site or preceding the protease cleavage site by no more than ten amino acid residues, wherein the protease cleavage site immediately precedes the amino acid tag or precedes the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the protease cleavage site, wherein the protease cleavage site immediately precedes the amino acid tag, and wherein an alanine immediately follows the amino acid tag. The capsid polypeptide of the second AAV can comprise a threonine and glycine (TG) sequence immediately preceding the protease cleavage site, wherein the protease cleavage site immediately precedes the amino acid tag, and wherein a glycine, leucine, and serine (GLS) sequence immediately follows the amino acid tag. The capsid polypeptide of the second AAV can comprise an N-terminal linker immediately preceding the amino acid tag or preceding the amino acid tag by no more than ten amino acid residues, wherein the protease cleavage site immediately follows the amino acid tag or follows the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, wherein the protease cleavage site immediately follows the amino acid tag, and wherein an alanine immediately follows the protease cleavage site. The capsid polypeptide of the second AAV can comprise a threonine and glycine (TG) sequence immediately preceding the amino acid tag, wherein the protease cleavage site immediately follows the amino acid tag, and wherein a glycine, leucine, and serine (GLS) sequence immediately follows the protease cleavage site. The capsid polypeptide of the second AAV can comprise a C-terminal linker immediately following the protease cleavage site or following the protease cleavage site by no more than ten amino acid residues, and wherein the protease cleavage site immediately follows the amino acid tag or follows the amino acid tag by no more than ten amino acid residues. The C-terminal linker can comprise (a) an alanine (A) or (b) a glycine, leucine, and serine (GLS) sequence. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The second AAV can be an AAV2.

In some cases, the conjugating polypeptide can be a conjugating polypeptide comprising (or consisting essentially of or consisting of) (a) a first binding motif and (b) a second binding motif, wherein the first binding motif comprises (i) the amino acid sequence set forth in SEQ ID NO:10 (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The first binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:11. The first binding motif can comprise the amino acid sequence set forth in SEQ ID NO:10, SEQ ID NO:26, or SEQ ID NO:27. The second binding motif can comprise (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The second binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:11. The second binding motif can comprise the amino acid sequence set forth in SEQ ID NO:10, SEQ ID NO:26, SEQ ID NO:27. The second binding motif can comprise (a) the amino acid sequence set forth in SEQ ID NO:18 or the amino acid sequence set forth in SEQ ID NO:18 with 25 or less amino acid additions, deletions, or substitutions, (b) the amino acid sequence set forth in SEQ ID NO:19 or the amino acid sequence set forth in SEQ ID NO:19 with 25 or less amino acid additions, deletions, or substitutions, or (c) the amino acid sequence set forth in SEQ ID NO:45 or the amino acid sequence set forth in SEQ ID NO:45 with 25 or less amino acid additions, deletions, or substitutions. The first binding motif and the second binding motif can be separated by a linker. The linker can comprise the amino acid sequence set forth in SEQ ID NO:41, SEQ ID NO:42, or SEQ ID NO:43. The conjugating polypeptide can comprise SEQ ID NO:41.

The method can comprise performing a purification procedure after the step (a) to form a composition enriched for the second AAV-conjugating polypeptide complex, wherein the composition enriched for the second AAV-conjugating polypeptide complex is used in the step (b). The method can comprise performing a purification procedure after the step (b) to form a composition enriched for the second AAV-conjugating polypeptide-first AAV complex.

In another aspect, this document features a method for producing a composition comprising (or consisting essentially of or consisting of) a first AAV covalently linked to a second AAV, wherein the first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, and wherein the second AAV comprises a capsid polypeptide comprising an amino acid tag. The method comprises (or consisting essentially of or consisting of) (a) contacting the first AAV with a conjugating polypeptide to form a first AAV-conjugating polypeptide complex, wherein the conjugating polypeptide comprises a first binding motif having the ability to bind covalently to the amino acid segment of the first AAV and a second binding motif having the ability to bind covalently to the amino acid tag of the second AAV, and (b) contacting the first AAV-conjugating polypeptide complex with the second AAV for form a first AAV-conjugating polypeptide-second AAV complex, thereby producing the composition comprising the first AAV covalently linked to the second AAV, wherein (i) the capsid polypeptide of the first AAV comprises a protease cleavage site within about 20 amino acid residues of the amino acid segment and the method comprises contacting the first AAV with a protease for the protease cleavage site of the first AAV prior to or during the step (a), (ii) the capsid polypeptide of the second AAV comprises a protease cleavage site within about 20 amino acid residues of the amino acid tag and the method comprises contacting the second AAV with a protease for the protease cleavage site of the second AAV prior to or during the step (b), or (iii) the capsid polypeptide of the first AAV comprises a protease cleavage site within about 20 amino acid residues of the amino acid segment, the method comprises contacting the first AAV with a protease for the protease cleavage site of the first AAV prior to or during the step (a), the capsid polypeptide of the second AAV comprises a protease cleavage site within about 20 amino acid residues of the amino acid tag, and the method comprises contacting the second AAV with a protease for the protease cleavage site of the second AAV prior to or during the step (b). The contacting step with the first AAV can comprise using a solution comprising a titer of the AAV that is greater than 1014.

In some cases, the first AAV can be an AAV comprising a capsid polypeptide, wherein the capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions. The AAV can be an AAV2. The amino acid segment can comprise the amino acid sequence set forth in SEQ ID NO:1 with three amino acid substitutions. The amino acid segment can comprise the amino acid sequence set forth in SEQ ID NO:1 with two amino acid substitutions. The amino acid segment can comprise the amino acid sequence set forth in SEQ ID NO:1 with one amino acid substitutions. The amino acid segment can comprise the amino acid sequence set forth in SEQ ID NO:1. The capsid polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The capsid polypeptide can comprise the amino acid segment at an amino acid position within three amino acid residues of an amino acid residue corresponding to position 453 of a reference VP1 polypeptide having the amino acid sequence set forth in SEQ ID NO:2. The capsid polypeptide can comprise the amino acid segment at an amino acid position within two amino acid residues of an amino acid residue corresponding to position 453 of a reference VP1 polypeptide having the amino acid sequence set forth in SEQ ID NO:2. The capsid polypeptide can comprise the amino acid segment at an amino acid position within one amino acid residue of an amino acid residue corresponding to position 453 of a reference VP1 polypeptide having the amino acid sequence set forth in SEQ ID NO:2. The capsid polypeptide can comprise the amino acid segment at an amino acid position of an amino acid residue corresponding to position 453 of a reference VP1 polypeptide having the amino acid sequence set forth in SEQ ID NO:2. The capsid polypeptide can comprise the amino acid segment at an amino acid position within three amino acid residues of an amino acid residue corresponding to position 588 of a reference VP1 polypeptide having the amino acid sequence set forth in SEQ ID NO:2. The capsid polypeptide can comprise the amino acid segment at an amino acid position within two amino acid residues of an amino acid residue corresponding to position 588 of a reference VP1 polypeptide having the amino acid sequence set forth in SEQ ID NO:2. The capsid polypeptide can comprise the amino acid segment at an amino acid position within one amino acid residue of an amino acid residue corresponding to position 588 of a reference VP1 polypeptide having the amino acid sequence set forth in SEQ ID NO:2. The capsid polypeptide can comprise the amino acid segment at an amino acid position of an amino acid residue corresponding to position 588 of a reference VP1 polypeptide having the amino acid sequence set forth in SEQ ID NO:2. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, and wherein the capsid polypeptide comprises an alanine immediately following the amino acid segment. The capsid polypeptide can comprise a threonine and glycine (TG) sequence immediately preceding the amino acid segment, and wherein the capsid polypeptide comprises a glycine, leucine, and serine (GLS) sequence immediately following the amino acid segment. The capsid polypeptide can comprise a protease cleavage site. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The protease cleavage site can immediately precede the amino acid segment or precedes the amino acid segment by no more than 20 amino acid residues. The protease cleavage site can precede the amino acid segment by no more than ten amino acid residues. The protease cleavage site can immediately follow the amino acid segment or follows the amino acid segment by no more than 20 amino acid residues. The protease cleavage site can follow the amino acid segment by no more than ten amino acid residues. The capsid polypeptide can comprise an N-terminal linker immediately preceding a protease cleavage site or preceding the protease cleavage site by no more than ten amino acid residues, wherein the protease cleavage site immediately precedes the amino acid segment or precedes the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the protease cleavage site, wherein the protease cleavage site immediately precedes the amino acid segment, and wherein an alanine immediately follows the amino acid segment. The capsid polypeptide can comprise a threonine and glycine (TG) sequence immediately preceding the protease cleavage site, wherein the protease cleavage site immediately precedes the amino acid segment, and wherein a glycine, leucine, and serine (GLS) sequence immediately follows the amino acid segment. The capsid polypeptide can comprise an N-terminal linker immediately preceding the amino acid segment or preceding the amino acid segment by no more than ten amino acid residues, and wherein the capsid polypeptide comprises a protease cleavage site immediately following the amino acid segment or following the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, wherein the protease cleavage site immediately follows the amino acid segment, and wherein an alanine immediately follows the protease cleavage site. The capsid polypeptide can comprise a threonine and glycine (TG) sequence immediately preceding the amino acid segment, wherein the protease cleavage site immediately follows the amino acid segment, and wherein a glycine, leucine, and serine (GLS) sequence immediately follows the protease cleavage site. The capsid polypeptide can comprise a C-terminal linker immediately following a protease cleavage site or following the protease cleavage site by no more than ten amino acid residues, and wherein the protease cleavage site immediately follows the amino acid segment or follows the amino acid segment by no more than ten amino acid residues. The C-terminal linker can comprise (a) an alanine (A) or (b) a glycine, leucine, and serine (GLS) sequence. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25.

In some cases, the second AAV can be as described in this paragraph. For example, the capsid polypeptides of the second AAV can lack the amino acid segment. The second AAV can comprise a capsid polypeptide comprising an amino acid tag comprising: (a) the amino acid sequence set forth in SEQ ID NO:1 or the amino acid sequence set forth in SEQ ID NO:1 with one, two, or three amino acid substitutions, (b) the amino acid sequence set forth in SEQ ID NO:8 or the amino acid sequence set forth in SEQ ID NO:8 with one, two, or three amino acid substitutions, (c) the amino acid sequence set forth in SEQ ID NO:9 or the amino acid sequence set forth in SEQ ID NO:9 with one, two, or three amino acid substitutions, or (d) the amino acid sequence set forth in SEQ ID NO:44 or the amino acid sequence set forth in SEQ ID NO:44 with one, two, or three amino acid substitutions. The capsid polypeptide of the second AAV can comprise the amino acid tag at an amino acid position within three amino acid residues of an amino acid residue corresponding to position 453 or position 588 of a reference VP1 polypeptide having the amino acid sequence set forth in SEQ ID NO:2. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, and wherein the capsid polypeptide of the second AAV comprises an alanine immediately following the amino acid tag. The capsid polypeptide of the second AAV can comprise a threonine and glycine (TG) sequence immediately preceding the amino acid tag, and wherein the capsid polypeptide of the second AAV comprises a glycine, leucine, and serine (GLS) sequence immediately following the amino acid tag. The capsid polypeptide of the second AAV can comprise a protease cleavage site. The capsid polypeptide of the second AAV can comprise a protease cleavage site. The protease cleavage site of the second AAV can immediately precede the amino acid tag or precedes the amino acid tag by no more than 20 amino acid residues. The protease cleavage site of the second AAV can precede the amino acid tag by no more than ten amino acid residues. The protease cleavage site of the second AAV can immediately follow the amino acid tag or follows the amino acid tag by no more than 20 amino acid residues. The protease cleavage site of the second AAV can follow the amino acid tag by no more than ten amino acid residues. The capsid polypeptide of the second AAV can comprise an N-terminal linker immediately preceding the protease cleavage site or preceding the protease cleavage site by no more than ten amino acid residues, wherein the protease cleavage site immediately precedes the amino acid tag or precedes the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the protease cleavage site, wherein the protease cleavage site immediately precedes the amino acid tag, and wherein an alanine immediately follows the amino acid tag. The capsid polypeptide of the second AAV can comprise a threonine and glycine (TG) sequence immediately preceding the protease cleavage site, wherein the protease cleavage site immediately precedes the amino acid tag, and wherein a glycine, leucine, and serine (GLS) sequence immediately follows the amino acid tag. The capsid polypeptide of the second AAV can comprise an N-terminal linker immediately preceding the amino acid tag or preceding the amino acid tag by no more than ten amino acid residues, wherein the protease cleavage site immediately follows the amino acid tag or follows the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, wherein the protease cleavage site immediately follows the amino acid tag, and wherein an alanine immediately follows the protease cleavage site. The capsid polypeptide of the second AAV can comprise a threonine and glycine (TG) sequence immediately preceding the amino acid tag, wherein the protease cleavage site immediately follows the amino acid tag, and wherein a glycine, leucine, and serine (GLS) sequence immediately follows the protease cleavage site. The capsid polypeptide of the second AAV can comprise a C-terminal linker immediately following the protease cleavage site or following the protease cleavage site by no more than ten amino acid residues, and wherein the protease cleavage site immediately follows the amino acid tag or follows the amino acid tag by no more than ten amino acid residues. The C-terminal linker can comprise (a) an alanine (A) or (b) a glycine, leucine, and serine (GLS) sequence. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The second AAV can be an AAV2.

In some cases, the conjugating polypeptide can be a conjugating polypeptide comprising (or consisting essentially of or consisting of) (a) a first binding motif and (b) a second binding motif, wherein the first binding motif comprises (i) the amino acid sequence set forth in SEQ ID NO:10 (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The first binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:11. The first binding motif can comprise the amino acid sequence set forth in SEQ ID NO:10, SEQ ID NO:26, or SEQ ID NO:27. The second binding motif can comprise (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The second binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:11. The second binding motif can comprise the amino acid sequence set forth in SEQ ID NO:10, SEQ ID NO:26, SEQ ID NO:27. The second binding motif can comprise (a) the amino acid sequence set forth in SEQ ID NO:18 or the amino acid sequence set forth in SEQ ID NO:18 with 25 or less amino acid additions, deletions, or substitutions, (b) the amino acid sequence set forth in SEQ ID NO:19 or the amino acid sequence set forth in SEQ ID NO:19 with 25 or less amino acid additions, deletions, or substitutions, or (c) the amino acid sequence set forth in SEQ ID NO:45 or the amino acid sequence set forth in SEQ ID NO:45 with 25 or less amino acid additions, deletions, or substitutions. The first binding motif and the second binding motif can be separated by a linker. The linker can comprise the amino acid sequence set forth in SEQ ID NO:41, SEQ ID NO:42, or SEQ ID NO:43. The conjugating polypeptide can comprise SEQ ID NO:41.

The method can comprise performing a purification procedure after the step (a) to form a composition enriched for the first AAV-conjugating polypeptide complex, wherein the composition enriched for the first AAV-conjugating polypeptide complex is used in the step (b). The method can comprise performing a purification procedure after the step (b) to form a composition enriched for the first AAV-conjugating polypeptide-second AAV complex.

In another aspect, this document features a method for producing a composition comprising (or consisting essentially of or consisting of) a first AAV covalently linked to a second AAV, wherein the first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, wherein the second AAV comprises a capsid polypeptide comprising an amino acid tag. The method comprises (or consisting essentially of or consisting of) (a) contacting the second AAV with a conjugating polypeptide to form a second AAV-conjugating polypeptide complex, wherein the conjugating polypeptide comprises a first binding motif having the ability to bind covalently to the amino acid segment of the first AAV and a second binding motif having the ability to bind covalently to the amino acid tag of the second AAV, and (b) contacting the second AAV-conjugating polypeptide complex with the first AAV for form a second AAV-conjugating polypeptide-first AAV complex, thereby producing the composition comprising the first AAV covalently linked to the second AAV, wherein (i) the capsid polypeptide of the first AAV comprises a protease cleavage site within about 20 amino acid residues of the amino acid segment and the method comprises contacting the first AAV with a protease for the protease cleavage site of the first AAV prior to or during the step (b), (ii) the capsid polypeptide of the second AAV comprises a protease cleavage site within about 20 amino acid residues of the amino acid tag and the method comprises contacting the second AAV with a protease for the protease cleavage site of the second AAV prior to or during the step (a), or (iii) the capsid polypeptide of the first AAV comprises a protease cleavage site within about 20 amino acid residues of the amino acid segment, the method comprises contacting the first AAV with a protease for the protease cleavage site of the first AAV prior to or during the step (b), the capsid polypeptide of the second AAV comprises a protease cleavage site within about 20 amino acid residues of the amino acid tag, and the method comprises contacting the second AAV with a protease for the protease cleavage site of the second AAV prior to or during the step (a). The contacting step with the first AAV can comprise using a solution comprising a titer of the AAV that is greater than 10¹⁴.

In some cases, the first AAV can be an AAV comprising a capsid polypeptide, wherein the capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions. The AAV can be an AAV2. The amino acid segment can comprise the amino acid sequence set forth in SEQ ID NO:1 with three amino acid substitutions. The amino acid segment can comprise the amino acid sequence set forth in SEQ ID NO:1 with two amino acid substitutions. The amino acid segment can comprise the amino acid sequence set forth in SEQ ID NO:1 with one amino acid substitutions. The amino acid segment can comprise the amino acid sequence set forth in SEQ ID NO:1. The capsid polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The capsid polypeptide can comprise the amino acid segment at an amino acid position within three amino acid residues of an amino acid residue corresponding to position 453 of a reference VP1 polypeptide having the amino acid sequence set forth in SEQ ID NO:2. The capsid polypeptide can comprise the amino acid segment at an amino acid position within two amino acid residues of an amino acid residue corresponding to position 453 of a reference VP1 polypeptide having the amino acid sequence set forth in SEQ ID NO:2. The capsid polypeptide can comprise the amino acid segment at an amino acid position within one amino acid residue of an amino acid residue corresponding to position 453 of a reference VP1 polypeptide having the amino acid sequence set forth in SEQ ID NO:2. The capsid polypeptide can comprise the amino acid segment at an amino acid position of an amino acid residue corresponding to position 453 of a reference VP1 polypeptide having the amino acid sequence set forth in SEQ ID NO:2. The capsid polypeptide can comprise the amino acid segment at an amino acid position within three amino acid residues of an amino acid residue corresponding to position 588 of a reference VP1 polypeptide having the amino acid sequence set forth in SEQ ID NO:2. The capsid polypeptide can comprise the amino acid segment at an amino acid position within two amino acid residues of an amino acid residue corresponding to position 588 of a reference VP1 polypeptide having the amino acid sequence set forth in SEQ ID NO:2. The capsid polypeptide can comprise the amino acid segment at an amino acid position within one amino acid residue of an amino acid residue corresponding to position 588 of a reference VP1 polypeptide having the amino acid sequence set forth in SEQ ID NO:2. The capsid polypeptide can comprise the amino acid segment at an amino acid position of an amino acid residue corresponding to position 588 of a reference VP1 polypeptide having the amino acid sequence set forth in SEQ ID NO:2. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, and wherein the capsid polypeptide comprises an alanine immediately following the amino acid segment. The capsid polypeptide can comprise a threonine and glycine (TG) sequence immediately preceding the amino acid segment, and wherein the capsid polypeptide comprises a glycine, leucine, and serine (GLS) sequence immediately following the amino acid segment. The capsid polypeptide can comprise a protease cleavage site. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The protease cleavage site can immediately precede the amino acid segment or precedes the amino acid segment by no more than 20 amino acid residues. The protease cleavage site can precede the amino acid segment by no more than ten amino acid residues. The protease cleavage site can immediately follow the amino acid segment or follows the amino acid segment by no more than 20 amino acid residues. The protease cleavage site can follow the amino acid segment by no more than ten amino acid residues. The capsid polypeptide can comprise an N-terminal linker immediately preceding a protease cleavage site or preceding the protease cleavage site by no more than ten amino acid residues, wherein the protease cleavage site immediately precedes the amino acid segment or precedes the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the amino acid segment or following the amino acid segment by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the protease cleavage site, wherein the protease cleavage site immediately precedes the amino acid segment, and wherein an alanine immediately follows the amino acid segment. The capsid polypeptide can comprise a threonine and glycine (TG) sequence immediately preceding the protease cleavage site, wherein the protease cleavage site immediately precedes the amino acid segment, and wherein a glycine, leucine, and serine (GLS) sequence immediately follows the amino acid segment. The capsid polypeptide can comprise an N-terminal linker immediately preceding the amino acid segment or preceding the amino acid segment by no more than ten amino acid residues, and wherein the capsid polypeptide comprises a protease cleavage site immediately following the amino acid segment or following the amino acid segment by no more than ten amino acid residues. The N-terminal linker can comprise (a) a leucine and alanine (LA) sequence or (b) a threonine and glycine (TG) sequence. The capsid polypeptide can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide can comprise a leucine and alanine (LA) sequence immediately preceding the amino acid segment, wherein the protease cleavage site immediately follows the amino acid segment, and wherein an alanine immediately follows the protease cleavage site. The capsid polypeptide can comprise a threonine and glycine (TG) sequence immediately preceding the amino acid segment, wherein the protease cleavage site immediately follows the amino acid segment, and wherein a glycine, leucine, and serine (GLS) sequence immediately follows the protease cleavage site. The capsid polypeptide can comprise a C-terminal linker immediately following a protease cleavage site or following the protease cleavage site by no more than ten amino acid residues, and wherein the protease cleavage site immediately follows the amino acid segment or follows the amino acid segment by no more than ten amino acid residues. The C-terminal linker can comprise (a) an alanine (A) or (b) a glycine, leucine, and serine (GLS) sequence. The capsid polypeptide can comprise an N-terminal linker immediately preceding the amino acid segment or preceding the amino acid segment by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25.

In some cases, the second AAV can be as described in this paragraph. For example, the capsid polypeptides of the second AAV can lack the amino acid segment. The second AAV can comprise a capsid polypeptide comprising an amino acid tag comprising: (a) the amino acid sequence set forth in SEQ ID NO:1 or the amino acid sequence set forth in SEQ ID NO:1 with one, two, or three amino acid substitutions, (b) the amino acid sequence set forth in SEQ ID NO:8 or the amino acid sequence set forth in SEQ ID NO:8 with one, two, or three amino acid substitutions, (c) the amino acid sequence set forth in SEQ ID NO:9 or the amino acid sequence set forth in SEQ ID NO:9 with one, two, or three amino acid substitutions, or (d) the amino acid sequence set forth in SEQ ID NO:44 or the amino acid sequence set forth in SEQ ID NO:44 with one, two, or three amino acid substitutions. The capsid polypeptide of the second AAV can comprise the amino acid tag at an amino acid position within three amino acid residues of an amino acid residue corresponding to position 453 or position 588 of a reference VP1 polypeptide having the amino acid sequence set forth in SEQ ID NO:2. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, and wherein the capsid polypeptide of the second AAV comprises an alanine immediately following the amino acid tag. The capsid polypeptide of the second AAV can comprise a threonine and glycine (TG) sequence immediately preceding the amino acid tag, and wherein the capsid polypeptide of the second AAV comprises a glycine, leucine, and serine (GLS) sequence immediately following the amino acid tag. The capsid polypeptide of the second AAV can comprise a protease cleavage site. The capsid polypeptide of the second AAV can comprise a protease cleavage site. The protease cleavage site of the second AAV can immediately precede the amino acid tag or precedes the amino acid tag by no more than 20 amino acid residues. The protease cleavage site of the second AAV can precede the amino acid tag by no more than ten amino acid residues. The protease cleavage site of the second AAV can immediately follow the amino acid tag or follows the amino acid tag by no more than 20 amino acid residues. The protease cleavage site of the second AAV can follow the amino acid tag by no more than ten amino acid residues. The capsid polypeptide of the second AAV can comprise an N-terminal linker immediately preceding the protease cleavage site or preceding the protease cleavage site by no more than ten amino acid residues, wherein the protease cleavage site immediately precedes the amino acid tag or precedes the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the amino acid tag or following the amino acid tag by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the protease cleavage site, wherein the protease cleavage site immediately precedes the amino acid tag, and wherein an alanine immediately follows the amino acid tag. The capsid polypeptide of the second AAV can comprise a threonine and glycine (TG) sequence immediately preceding the protease cleavage site, wherein the protease cleavage site immediately precedes the amino acid tag, and wherein a glycine, leucine, and serine (GLS) sequence immediately follows the amino acid tag. The capsid polypeptide of the second AAV can comprise an N-terminal linker immediately preceding the amino acid tag or preceding the amino acid tag by no more than ten amino acid residues, wherein the protease cleavage site immediately follows the amino acid tag or follows the amino acid tag by no more than ten amino acid residues. The N-terminal linker can comprise (a) a leucine and alanine (LA) sequence or (b) a threonine and glycine (TG) sequence. The capsid polypeptide of the second AAV can comprise a C-terminal linker immediately following the protease cleavage site or following the protease cleavage site by no more than ten amino acid residues. The C-terminal linker can comprise (a) an alanine (A) or (b) a glycine, leucine, and serine (GLS) sequence. The capsid polypeptide of the second AAV can comprise a leucine and alanine (LA) sequence immediately preceding the amino acid tag, wherein the protease cleavage site immediately follows the amino acid tag, and wherein an alanine immediately follows the protease cleavage site. The capsid polypeptide of the second AAV can comprise a threonine and glycine (TG) sequence immediately preceding the amino acid tag, wherein the protease cleavage site immediately follows the amino acid tag, and wherein a glycine, leucine, and serine (GLS) sequence immediately follows the protease cleavage site. The capsid polypeptide of the second AAV can comprise a C-terminal linker immediately following the protease cleavage site or following the protease cleavage site by no more than ten amino acid residues, and wherein the protease cleavage site immediately follows the amino acid tag or follows the amino acid tag by no more than ten amino acid residues. The C-terminal linker can comprise (a) an alanine (A) or (b) a glycine, leucine, and serine (GLS) sequence. The capsid polypeptide of the second AAV can comprise an N-terminal linker immediately preceding the amino acid tag or preceding the amino acid tag by no more than ten amino acid residues. The N-terminal linker can comprise (a) a leucine and alanine (LA) sequence or (b) a threonine and glycine (TG) sequence. The protease cleavage site can be a cleavage site for a protease selected from the group consisting of a TEV protease, a PreScission protease, thrombin, Factor Xa, and enterokinase. The protease cleavage site can comprise the amino acid sequence set forth in any one of SEQ ID NOs:20-25. The second AAV can be an AAV2.

In some cases, the conjugating polypeptide can be a conjugating polypeptide comprising (or consisting essentially of or consisting of) (a) a first binding motif and (b) a second binding motif, wherein the first binding motif comprises (i) the amino acid sequence set forth in SEQ ID NO:10 (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The first binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The first binding motif can comprise the amino acid sequence set forth in SEQ ID NO:11. The first binding motif can comprise the amino acid sequence set forth in SEQ ID NO:10, SEQ ID NO:26, or SEQ ID NO:27. The second binding motif can comprise (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27. The second binding motif can comprise the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid. The second binding motif can comprise the amino acid sequence set forth in SEQ ID NO:11. The second binding motif can comprise the amino acid sequence set forth in SEQ ID NO:10, SEQ ID NO:26, SEQ ID NO:27. The second binding motif can comprise (a) the amino acid sequence set forth in SEQ ID NO:18 or the amino acid sequence set forth in SEQ ID NO:18 with 25 or less amino acid additions, deletions, or substitutions, (b) the amino acid sequence set forth in SEQ ID NO:19 or the amino acid sequence set forth in SEQ ID NO:19 with 25 or less amino acid additions, deletions, or substitutions, or (c) the amino acid sequence set forth in SEQ ID NO:45 or the amino acid sequence set forth in SEQ ID NO:45 with 25 or less amino acid additions, deletions, or substitutions. The first binding motif and the second binding motif can be separated by a linker. The linker can comprise the amino acid sequence set forth in SEQ ID NO:41, SEQ ID NO:42, or SEQ ID NO:43. The conjugating polypeptide can comprise SEQ ID NO:41.

The method can comprise performing a purification procedure after the step (a) to form a composition enriched for the second AAV-conjugating polypeptide complex, wherein the composition enriched for the second AAV-conjugating polypeptide complex is used in the step (b). The method can comprise performing a purification procedure after the step (b) to form a composition enriched for the second AAV-conjugating polypeptide-first AAV complex.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic of two AAVs covalently attached together according to one embodiment.

FIG. 2 is a sequence listing of the amino acid sequence (SEQ ID NO:2) of a reference VP1 capsid polypeptide of AAV2.*=position 588 for possible insertion.

FIGS. 3A-3B are images of green fluorescent protein (GFP) in HEK 293 cells following infection with AAV2-588-TG-SpyTag-GLS (FIG. 3A) and AAV2-588-TG-AAS-GLS (FIG. 3B).

FIG. 4 is a schematic of a second AAV including a capsid polypeptide containing a binding moiety. Protease cleavage of a first AAV releases one end of the amino acid segment, and a covalently bond between the amino acid segment and the binding moiety binds the first AAV and the second AAV together.

FIG. 5 is a set of images of GFP fluorescence in HEK 293 cells following infection with AAV-SpyTag capsid polypeptides having a tev cleavage site in addition to the SpyTag inserted at amino acid position 588.

FIG. 6 is a sequence listing of the amino acid sequence of AAV2-588-LA-AAS-A.

FIG. 7 is a sequence listing of the amino acid sequence of AAV2-588-TG-AAS-GLS.

FIG. 8 is a sequence listing of the amino acid sequence of AAV2-588-LA-tev-SpyTag-A.

FIG. 9 is a sequence listing of the amino acid sequence of AAV2-588-TG-tev-Spy Tag-GLS.

### DETAILED DESCRIPTION

This document provides AAVs containing a capsid polypeptide that includes an amino acid segment containing the amino acid sequence set forth in SEQ ID NO:1 (or a variant thereof). An appropriate AAV can be engineered to contain a capsid polypeptide that includes an amino acid segment containing the amino acid sequence set forth in SEQ ID NO:1 (or a variant thereof). For example, AAV1, AAV2, AAV4, AAVS, AAV6, AAV7, AAV8, and AAV9 can be engineered to contain a capsid polypeptide that includes an amino acid segment containing the amino acid sequence set forth in SEQ ID NO:1 (or a variant thereof). In some cases, an AAV2 can be engineered to include a capsid polypeptide that includes an amino acid segment containing the amino acid sequence set forth in SEQ ID NO:1.

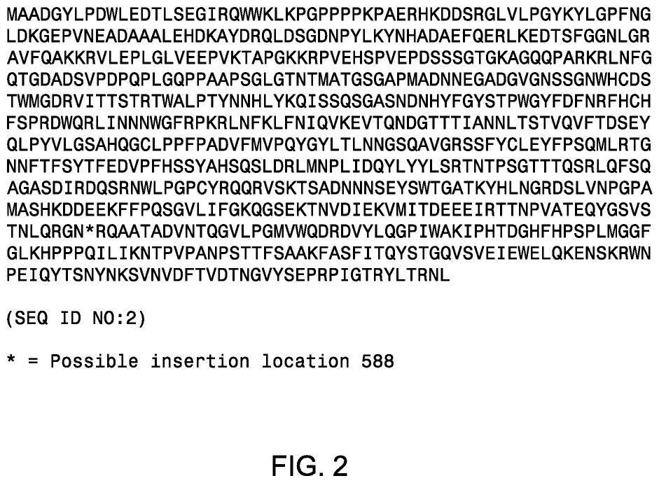

As described herein, an AAV (e.g., AAV2) can be designed such that the amino acid sequence set forth in SEQ ID NO:1 (or a variant thereof) is inserted into a capsid polypeptide of that AAV. Any appropriate site of a capsid polypeptide (e.g., a VP1, VP2, and/or VP3 capsid polypeptide) can be used as the site for accommodating insertion of the amino acid sequence set forth in SEQ ID NO:1 (or a variant thereof). Examples of such sites include, without limitation, those amino acid positions of a VP3 polypeptide that correspond to amino acid position 262, amino acid position 453, amino acid position 587, amino acid position 588, and amino acid position 589 of a reference VP1 polypeptide with the amino acid sequence set forth in SEQ ID NO:2 (FIG. 2). The numbering of VP1 is used with respect to VP1, VP2, and VP3 as VP2 and VP3 are polypeptides that use alternative start sites within VP1. In one example, an AAV2 can be engineered to include a capsid polypeptide that includes an amino acid segment containing the amino acid sequence set forth in SEQ ID NO:1 located at position 588 of a VP3 polypeptide (which is based on the VP1 amino acid numbering) of that AAV2.

As described herein, an amino acid segment that contains the amino acid sequence set forth in SEQ ID NO:1 or a variant of the amino acid sequence set forth in SEQ ID NO:1 can be inserted into a capsid polypeptide (e.g., a VP3 polypeptide) of an AAV. Any appropriate variant of the amino acid sequence set forth in SEQ ID NO:1 can be used. For example, a variant of the amino acid sequence set forth in SEQ ID NO:1 can be designed to include one, two, three, four, five, six, or seven amino acid additions, deletions, or substitutions and can be used as described herein. In some cases, a variant of the amino acid sequence set forth in SEQ ID NO:1 can be designed to include one, two, or three amino acid additions, deletions, or substitutions. Examples of variants of the amino acid sequence set forth in SEQ ID NO:1 that can be used as described herein include, without limitation, those set forth in Table 1.

TABLE 1
Examples of variant of SEQ ID NO: 1.

| Variant | | |
|---|---|---|
| Number | Sequence | SEQ ID NO: |
| 1 | APIVMIDNDKPIT | 3 |
| 2 | DHIVMIDNDKPIT | 4 |
| 3 | DPIVMVDNDKPIT | 5 |
| 4 | DPIVMIDADKPIT | 6 |
| 5 | DPIVMIDNYKPIT | 7 |
| 6 | DPIVMIDNDKPYT | 36 |
| 7 | DPIVMIDNDKPIT | 37 |
| 8 | GSDPIVMIDNDKPITGS | 38 |
| 9 | PIVMIDNDKPIT | 39 |
| 10 | PIVMIDNDKPI | 40 |

In some cases, an AAV described herein can be designed to include one or more linkers within a capsid polypeptide. For example, an AAV described herein can be designed to include (a) a linker located N-terminal to the amino acid segment containing the amino acid sequence set forth in SEQ ID NO:1 (or a variant thereof), (b) a linker located C-terminal to the amino acid segment containing the amino acid sequence set forth in SEQ ID NO:1 (or a variant thereof), or (c) a linker located N-terminal and a linker located C-terminal to the amino acid segment containing the amino acid sequence set forth in SEQ ID NO:1 (or a variant thereof). Such linkers can be immediately adjacent to the amino acid segment containing the amino acid sequence set forth in SEQ ID NO:1 (or a variant thereof) or can be within 10, 15, 20, 25, or 30 amino acid residues of the amino acid segment containing the amino acid sequence set forth in SEQ ID NO:1 (or a variant thereof). For example, an AAV described herein can be designed to include, within a capsid polypeptide (e.g., a VP3 polypeptide) in an N-terminal to C-terminal direction, a capsid amino acid sequence immediately followed by a first linker immediately followed by the amino acid sequence set forth in SEQ ID NO:1 immediately followed by a second linker immediately followed a continuation of the capsid amino acid sequence. In another example, an AAV described herein can be designed to include, within a capsid polypeptide (e.g., a VP3 polypeptide) in an N-terminal to C-terminal direction, a capsid amino acid sequence immediately followed by a first linker followed within 10 amino acid residues by the amino acid sequence set forth in SEQ ID NO:1 followed within 10 amino acid residues by a second linker immediately followed a continuation of the capsid amino acid sequence. In another example, an AAV described herein can be designed to include, within a capsid polypeptide (e.g., a VP3 polypeptide) in an N-terminal to C-terminal direction, a capsid amino acid sequence immediately followed by a first linker immediately followed by a continuation of the capsid amino acid sequence that is less than 20 amino acid residues in length immediately followed by the amino acid sequence set forth in SEQ ID NO:1 immediately followed a continuation of the capsid amino acid sequence that is less than 20 amino acid residues in length immediately followed by a second linker immediately followed a continuation of the capsid amino acid sequence.

Any appropriate amino acid or amino acid sequence can be used as a linker to flank (e.g., immediately flank or flank within 10, 15, 20, 25, or 30 amino acid residues) one or both ends of an amino acid segment described herein (e.g., an amino acid segment containing the amino acid sequence set forth in SEQ ID NO:1 (or a variant thereof)) that is inserted into a capsid polypeptide. In some cases, a single amino acid can be used as a linker. Examples of linkers that can be used to flank (e.g., immediately flank or flank within 10, 15, 20, 25, or 30 amino acid residues) one or both ends of an amino acid segment containing the amino acid sequence set forth in SEQ ID NO:1 (or a variant thereof) include, without limitation, leucine-alanine (LA), alanine (A), threonine-glycine (TG), glycine-leucine-serine (GLS), glycine-glycine-serine (GGS), and glycine-serine (GS).

When an AAV provided herein is designed to include two or more linkers within a capsid polypeptide those linkers can be the same or different. For example, an AAV provided herein can be designed to include a capsid polypeptide that contains (a) a GLS linker N-terminal to (e.g., immediately N-terminal to or within 10, 15, 20, 25, or 30 amino acid residues to) an amino acid segment containing the amino acid sequence set forth in SEQ ID NO:1 (or a variant thereof) and (b) a GLS linker C-terminal to (e.g., immediately C-terminal to or within 10, 15, 20, 25, or 30 amino acid residues to) the amino acid segment containing the amino acid sequence set forth in SEQ ID NO:1 (or a variant thereof). In another example, an AAV provided herein can be designed to include a capsid polypeptide that contains (a) an LA linker N-terminal to (e.g., immediately N-terminal to or within 10, 15, 20, 25, or 30 amino acid residues to) an amino acid segment containing the amino acid sequence set forth in SEQ ID NO:1 (or a variant thereof) and (b) an A linker C-terminal to (e.g., immediately C-terminal to or within 10, 15, 20, 25, or 30 amino acid residues to) the amino acid segment containing the amino acid sequence set forth in SEQ ID NO:1 (or a variant thereof). In another example, an AAV provided herein can be designed to include a capsid polypeptide that contains (a) an TG linker N-terminal to (e.g., immediately N-terminal to or within 10, 15, 20, 25, or 30 amino acid residues to) an amino acid segment containing the amino acid sequence set forth in SEQ ID NO:1 (or a variant thereof) and (b) an GLS linker C-terminal to (e.g., immediately C-terminal to or within 10, 15, 20, 25, or 30 amino acid residues to) the amino acid segment containing the amino acid sequence set forth in SEQ ID NO:1 (or a variant thereof).

In some cases, an AAV described herein can be designed to include one or more (e.g., one, two, three, four, or five) protease cleavage sites within a capsid polypeptide. For example, an AAV described herein can be designed to include (a) one or more protease cleavage sites located within a capsid polypeptide N-terminal to the amino acid segment containing the amino acid sequence set forth in SEQ ID NO:1 (or a variant thereof), or (b) one or more protease cleavage sites located within a capsid polypeptide C-terminal to the amino acid segment containing the amino acid sequence set forth in SEQ ID NO:1 (or a variant thereof). Typically, the one or more protease cleavage sites are designed to be located within a capsid polypeptide either N-terminal to the amino acid segment containing the amino acid sequence set forth in SEQ ID NO:1 (or a variant thereof) or C-terminal to the amino acid segment containing the amino acid sequence set forth in SEQ ID NO:1 (or a variant thereof), but not both. For example, when an AAV described herein is designed to include one or more protease cleavage sites located within a capsid polypeptide N-terminal to the amino acid segment containing the amino acid sequence set forth in SEQ ID NO:1 (or a variant thereof), the capsid polypeptide can lack those protease cleavage sites C-terminal to the amino acid sequence set forth in SEQ ID NO:1 (or a variant thereof). Likewise, when an AAV described herein is designed to include one or more protease cleavage sites located within a capsid polypeptide C-terminal to the amino acid segment containing the amino acid sequence set forth in SEQ ID NO:1 (or a variant thereof), the capsid polypeptide can lack those protease cleavage sites N-terminal to the amino acid sequence set forth in SEQ ID NO:1 (or a variant thereof).

In some cases, one or more protease cleavage sites can be engineered into a capsid polypeptide described herein such that the site(s) are located immediately adjacent to (either N-terminal to or C-terminal to) the amino acid segment containing the amino acid sequence set forth in SEQ ID NO:1 (or a variant thereof) or can be within 10, 15, 20, 25, or 30 amino acid residues of (either N-terminal to or C-terminal to) the amino acid segment containing the amino acid sequence set forth in SEQ ID NO:1 (or a variant thereof). For example, an AAV described herein can be designed to include, within a capsid polypeptide (e.g., a VP3 polypeptide) in an N-terminal to C-terminal direction, a capsid amino acid sequence immediately followed by one or more protease cleavage sites immediately followed by the amino acid sequence set forth in SEQ ID NO:1 (or a variant thereof) immediately followed by a continuation of the capsid amino acid sequence. In another example, an AAV described herein can be designed to include, within a capsid polypeptide (e.g., a VP3 polypeptide) in an N-terminal to C-terminal direction, a capsid amino acid sequence immediately followed by the amino acid sequence set forth in SEQ ID NO:1 (or a variant thereof) immediately followed by one or more protease cleavage sites immediately followed by a continuation of the capsid amino acid sequence.

Any appropriate protease cleavage site can be used to create an AAV described herein. For example, the following protease cleavage site of a TEV protease can be used as described herein: an ENLYFQ⬇G sequence (SEQ ID NO:20). Other examples of protease cleavage sites (with their corresponding protease) include, without limitation, those set forth in Table 2.

TABLE 2
Examples of protease cleavage sites.

| Protease | Protease Cleavage Site Sequence | SEQ ID NO: |
|---|---|---|
| PreScission | LEVLF(Q/G)P | 21 |
| Thrombin | LVPRGS | 22 |
| Factor Xa | I(E/P)GR | 23 |
| Enterokinase | DDDDK | 24 |
| CleanCut | LVPWELQ ↓ E | 25 |

In some cases, an AAV described herein can be designed to include one or more (e.g., one, two, three, four, or five) protease cleavage sites in combination with one or more linkers within a capsid polypeptide. Examples of combinations of protease cleavage sites and linkers that can be used to make an AAV described herein include, without limitation, those set forth in Table 3. Each dash located between two listed components in Table 3 can represent that those two components are either immediately contiguous or within 1, 2, 3, 4, 5, 6, 7, 8, 9, 10, 15, 20, 25, or 30 amino acid residues of each other. In some cases, when locate within 1, 2, 3, 4, 5, 6, 7, 8, 9, 10, 15, 20, 25, or 30 amino acid residues of each other, the intervening amino acid(s) can be of the capsid polypeptide.

TABLE 3
Examples of combinations of protease cleavage sites and linkers.

| Example | Order (N-terminal to C-terminal direction) |
|---|---|
| 1 | L-PCS-AAS |
| 2 | L-PCS-L-AAS |
| 3 | L-PCS-AAS-L |
| 4 | L-PCS-L-AAS-L |
| 5 | PCS-L-AAS |
| 6 | PCS-L-AAS-L |
| 7 | PCS-AAS-L |
| 8 | L-AAS-PCS |
| 9 | L-AAS-L-PCS |
| 10 | L-AAS-PCS-L |
| 11 | L-AAS-L-PCS-L |
| 12 | AAS-L-PCS |
| 13 | AAS-L-PCS-L |
| 14 | AAS-PCS-L |
| 15 | L-PCS-PCS-AAS |
| 16 | PCS-PCS-AAS-L |
| 17 | L-PCS-PCS-AAS-L |
| 18 | L-PCS-PCS-PCS-AAS |
| 19 | PCS-PCS-PCS-AAS-L |
| 20 | L-PCS-PCS-PCS-AAS-L |
| 21 | L-PCS-PCS-PCS-PCS-AAS |
| 22 | PCS-PCS-PCS-PCS-AAS-L |
| 23 | L-PCS-PCS-PCS-PCS-AAS-L |
| 24 | L-AAS-PCS-PCS |
| 25 | AAS-PCS-PCS-L |
| 26 | L-AAS-PCS-PCS-L |
| 27 | L-AAS-PCS-PCS-PCS |
| 28 | AAS-PCS-PCS-PCS-L |
| 29 | L-AAS-PCS-PCS-PCS-L |
| 30 | L-AAS-PCS-PCS-PCS-PCS |
| 31 | AAS-PCS-PCS-PCS-PCS-L |
| 32 | L-AAS-PCS-PCS-PCS-PCS-L |

Abbreviations for Table 3:
L = Linker
PCS = Protease Cleavage Site
AAS = Amino Acid Segment containing the amino acid sequence set forth in SEQ ID NO: 1 (or a variant thereof)

In some cases, an AAV described herein can be designed to include, within a capsid polypeptide (e.g., a VP3 polypeptide) in an N-terminal to C-terminal direction, a capsid amino acid sequence immediately followed by a first linker immediately followed by a protease cleavage site immediately followed by the amino acid sequence set forth in SEQ ID NO:1 (or a variant thereof) immediately followed by a second linker immediately followed a continuation of the capsid amino acid sequence. In another example, an AAV described herein can be designed to include, within a capsid polypeptide (e.g., a VP3 polypeptide) in an N-terminal to C-terminal direction, a capsid amino acid sequence immediately followed by a first linker followed within 10 amino acid residues by a protease cleavage site immediately followed by the amino acid sequence set forth in SEQ ID NO:1 (or a variant thereof) followed within 10 amino acid residues by a second linker immediately followed a continuation of the capsid amino acid sequence. In another example, an AAV described herein can be designed to include, within a capsid polypeptide (e.g., a VP3 polypeptide) in an N-terminal to C-terminal direction, a capsid amino acid sequence immediately followed by a first linker immediately followed by a continuation of the capsid amino acid sequence that is less than 20 amino acid residues in length immediately followed by a protease cleavage site immediately followed by the amino acid sequence set forth in SEQ ID NO:1 (or a variant thereof) immediately followed a continuation of the capsid amino acid sequence that is less than 20 amino acid residues in length immediately followed by a second linker immediately followed a continuation of the capsid amino acid sequence.

An AAV having a capsid polypeptide that includes an amino acid segment containing the amino acid sequence set forth in SEQ ID NO:1 (or a variant thereof) can have the ability to form a covalent bond with another moiety via that amino acid segment. For example, an AAV provided herein having a capsid polypeptide that includes an amino acid segment containing the amino acid sequence set forth in SEQ ID NO:1 (or a variant thereof) can covalently bind to a polypeptide (e.g., a conjugating polypeptide) or another virus (e.g., another AAV) via that amino acid segment. To form that covalent bond, the other moiety can be designed to include a binding moiety having the ability to form that covalent bond with the amino acid segment containing the amino acid sequence set forth in SEQ ID NO:1 (or a variant thereof).

A binding moiety having the ability to form a covalent bond with an amino acid segment containing the amino acid sequence set forth in SEQ ID NO:1 (or a variant thereof) that can be used as described herein can have any appropriate amino acid sequence. Examples of binding moieties having the ability to form a covalent bond with an amino acid segment containing the amino acid sequence set forth in SEQ ID NO:1 (or a variant thereof) that can be used as described herein include, without limitation, those set forth in the following formula:

DSTTH X₁ KFSKRDING X₂ ELAGA X₃ IELRN X₄ SG X₅
TIQSW X₆ SDG X₇ VKDFYLMPG X₈ YQFVETAAPEGYEL X₉
APITFTIDE X₁₀ GQIWV X₁₁ S

where X₁ to X₁₁=any of the twenty amino acids (SEQ ID NO:11).

Other examples of binding moieties having the ability to form a covalent bond with an amino acid segment containing the amino acid sequence set forth in SEQ ID NO:1 (or a variant thereof) that can be used as described herein include, without limitation, those set forth in the following formula, designated herein as SEQ ID NO:26:

DSTTH X₁ KFSKRDING X₂ ELAGA X₃ IELRN X₄ SG X₅
TIQSW X₆ SDG X₇ VK(D/V)FYLMPG X₈ YQFVETAAPEG
YEL X₉ APITFTIDE X₁₀ GQIWV X₁₁ S

where:

X₁=valine, alanine, isoleucine, or leucine;

X₂=lysine, arginine, histidine, or serine;

X₃=methionine, leucine, phenylalanine, or tryptophan

X₄=leucine, methionine, isoleucine, or tyrosine;

X₅=glutamine, serine, threonine, or asparagine;

X₆=isoleucine, leucine, methionine, or phenylalanine;

X₇=threonine, serine, valine, or asparagine;

X₈=threonine, serine, valine, or asparagine;

X₉=alanine, isoleucine, leucine, or phenylalanine;

X₁₀=lysine, arginine, histidine, or serine; and

X₁₁=aspartic acid, glutamic acid, or lysine.

Other examples of binding moieties having the ability to form a covalent bond with an amino acid segment that contains the amino acid sequence set forth in SEQ ID NO:1 (or a variant thereof) that can be used as described herein also include, without limitation, those set forth in the following formula:

IDT X₁ SGLS X₂ X₃ TX₄X₅S X₆ NTT X₇ EEDSTTH X₈
KFSKRDING X₉ ELAGA X₁₀ IELRN X₁₁ SG X₁₂
TIQSW X₁₃ SDG X₁₄ VK(D/V)FYLMPG X₁₅
YQFVETAAPEGYEL X₁₆ APITFTIDE X₁₇ GQIWV
X₁₈ S X₁₉ LIVGDDP X₂₀ VMIDNDKPIT

where X₁ to X₂₀=any of the twenty amino acids (SEQ ID NO:13).

Still other examples of binding moieties having the ability to form a covalent bond with an amino acid segment containing the amino acid sequence set forth in SEQ ID NO:1 (or a variant thereof) that can be used as described herein include, without limitation, those set forth in the following formula, designated herein as SEQ ID NO:27:

IDT X₁ SGLS X₂ X₃ TX₄X₅ S X₆ NTT X₇ EEDSTTH
X₈ KFSKRDING X₉ ELAGA X₁₀ IELRN X₁₁ SG
X₁₂ TIQSW X₁₃ SDG X₁₄ VK(D/V)FYLMPG X₁₅
YQFVETAAPEGYEL X₁₆ APITFTIDE X₁₇ GQIWV
X₁₈ S X₁₉ LIVGDDP X₂₀ VMIDNDKPIT

where

X₁=methionine, isoleucine, leucine, or phenylalanine;

X₂=glycine, proline, cysteine, or serine;

X₃=glutamic acid, aspartic acid, arginine, or lysine;

X₄=glycine, proline, cysteine, or serine;

X₅=glutamine, serine, threonine, or asparagine;

X₆=glycine, proline, cysteine, or serine;

X₇=isoleucine, leucine, methionine, or phenylalanine;

X₈=valine, alanine, isoleucine, or leucine;

X₉=lysine, arginine, histidine, or serine;

X₁₀=methionine, leucine, phenylalanine, or tryptophan;

X₁₁=leucine, methionine, isoleucine, or tyrosine;

X₁₂=glutamine, serine, threonine, or asparagine;

X₁₃=isoleucine, leucine, methionine, or phenylalanine;

X₁₄=threonine, serine, valine, or asparagine;

X₁₅=threonine, serine, valine, or asparagine;

X₁₆=alanine, isoleucine, leucine, or phenylalanine;

X₁₇=lysine, arginine, histidine, or serine;

X₁₈=aspartic acid, glutamic acid, or lysine;

X₁₉=threonine, serine, valine, or asparagine;

X₂₀=isoleucine, leucine, methionine, or phenylalanine.

In some cases, a binding moiety having the ability to form a covalent bond with an amino acid segment containing the amino acid sequence set forth in SEQ ID NO:1 (or a variant thereof) that can be used as described herein can have the amino acid sequence as set forth in any one of the sequences of Table 4 (any one of SEQ ID NOs:10, 12, and 14-17). In some cases, a binding moiety having the ability to form a covalent bond with an amino acid segment containing the amino acid sequence set forth in SEQ ID NO:1 (or a variant thereof) that can be used as described herein can have the amino acid sequence as set forth any one of SEQ ID NOs:10, 12, and 14-17 with 20 or less (e.g., one, two, three, four, five, six, seven, eight, nine, ten, 11, 12, 13, 14, 15, 16, 17, 18, 19, or 20) amino acid additions, deletions, or substitutions with the proviso that the underlined amino acid residue remains unchanged. For example, a binding moiety having the ability to form a covalent bond with an amino acid segment containing the amino acid sequence set forth in SEQ ID NO:1 (or a variant thereof) that can be used as described herein can have the amino acid sequence as set forth in SEQ ID NO:10 with one, two, three, four, or five amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.

TABLE 4
Examples of binding moieties
having the ability to form a
covalent bond with SEQ ID NO: 1
(or a variant thereof).

| | SEQ |
|---|---|
| | ID |
| Sequence | NO: |
| DSTTHVKFSKRDINGKELAGA | 10 |
| MIELRNLSGQTIQSWVSDGTV | |
| K(D/V)FYLMPGTYQFVETAA | |
| PEGYELAAPITFTIDEKGQIW | |
| VDS | |
| IDTMSGLSGETGQSGNTTIEE | 12 |
| DSTTHVKFSKRDINGKELAGA | |
| MIELRNLSGQTIQSWVSDGTV | |
| K(D/V)FYLMPGTYQFVETAA | |
| PEGYELAAPITFTIDEKGQIW | |
| VDSTLIVGDDPIVMIDNDKPI | |
| T | |
| DTMSGLSGETGQSGNTTIEED STTHVKFSKRDINGKELAGAM | 14 |
| IELRNLSGQTIQSWVSDGTVK | |
| (D/V)FYLMPGTYQFVETAAP | |
| EGYELAAPITFTIDEKGQIWV | |
| DSTLIVGDDP | |
| DTMSGLSGETGQSGNTTIEED STTHVKFSKRDINGKELAGAM | 15 |
| IELRNLSGQTIQSWVSDGTV | |
| K(D/V)FYLMPGTYQFVETA | |
| APEGYELAAPITFTIDEKGQ | |
| IWVDS | |
| DSTTHVKFSKRDINGKELAG | 16 |
| AMIELRNLSGQTIQSWVSDG | |
| TVK(D/V)FYLMPGTYQFVE | |
| TAAPEGYELAAPITFTIDEK | |
| GQIWVDSTLIVGDDPIVMID | |
| NDKPIT | |
| DTMSGLSGETGQSGNTTIEE | 17 |
| DSTTHVKFSKRDINGKELAG | |
| AMIELRNLSGQTIQSWVSDG | |
| TVK(D/V)FYLMPGTYQFVE | |
| TAAPEGYELAAPITFTIDEK | |
| GQIWVDSTLIVGDDPIVMID | |
| NDKPIT | |

As described herein, an AAV having a capsid polypeptide that includes an amino acid segment containing the amino acid sequence set forth in SEQ ID NO:1 (or a variant thereof) can be covalently bound to another moiety of interest via that amino acid segment. Any appropriate moiety of interest (e.g., a polypeptide or a virus) can be designed to include a binding moiety such that that moiety of interest can be covalently bound to an AAV provided herein via a covalent bond formed between the amino acid segment of the AAV and the binding moiety of the moiety of interest. Examples of moieties of interest that can be designed to include a binding moiety described herein and used as described herein include, without limitation, polypeptides such as a conjugating polypeptide, a Rabies G-protein, or a genome editing polypeptide and viruses such as AAVs or adenoviruses.

In some cases, a first AAV can be designed to include a capsid polypeptide having an amino acid segment containing the amino acid sequence set forth in SEQ ID NO:1 (or a variant thereof), and a second AAV can be designed to include a capsid polypeptide having a binding moiety having the ability to covalently bind to the amino acid segment of the first AAV. In such a case, the first AAV can be covalently bound to the second AAV via the amino acid segment of the first AAV and the binding moiety of the second AAV.

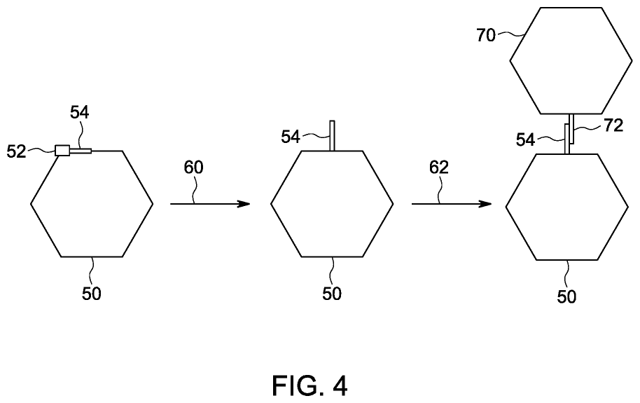

For example, with reference to FIG. 4, a first AAV (50) can be designed to include a capsid polypeptide containing an amino acid segment (54) containing the amino acid sequence set forth in SEQ ID NO:1 (or a variant thereof) as described herein. A second AAV (70) can be designed to include a capsid polypeptide containing a binding moiety (72). First AAV (50) and second AAV (70) can be combined to covalently link first AAV (50) to second AAV (70) via the covalent bond between amino acid segment (54) and binding moiety (72). In some examples, the covalently attached AAVs can enter a single cell as one unit (first AAV (50) and second AAV (70)), thereby delivering the nucleic acid content of the two AAVs to that cell.

In some cases, with further reference to FIG. 4, first AAV (50) can be designed to include a capsid polypeptide containing a protease cleavage site (52)N-terminal (or C-terminal) to amino acid segment (54) containing the amino acid sequence set forth in SEQ ID NO:1 (or a variant thereof) as described herein. Protease cleavage site (52) of first AAV (50) can be cleaved when exposed to a protease under conditions sufficient to allow proteolytic cleavage (60). Proteolytic cleavage (60) of protease cleavage site (52) can produce amino acid segment (54) where the N-terminal end (or C-terminal end) of amino acid segment (54) is released from the capsid polypeptide. Released amino acid segment (54) can more readily form a covalently bond with binding moiety (72) on second AAV (70).

In some cases, with further reference to FIG. 4, second AAV (70) can be designed to include a capsid polypeptide containing a protease cleavage site N-terminal (or C-terminal) to binding moiety (72). In these cases, proteolytic cleavage of the protease cleavage site can produce binding moiety (72) where the N-terminal end (or C-terminal end) of binding moiety (72) is released from the capsid polypeptide. A released binding moiety (72) can more readily form a covalent bond with amino acid segment (54).

AAVs (e.g., the “second AAV” of the preceding two paragraphs) having a capsid polypeptide designed to include a binding moiety having the ability to covalently bind to the amino acid segment of another AAV can be designed using any of the approaches described herein with respect to an amino acid segment containing the amino acid sequence set forth in SEQ ID NO:1 (or a variant thereof) with the binding moiety being used in place of the amino acid segment containing the amino acid sequence set forth in SEQ ID NO:1 (or a variant thereof). For example, an AAV having a capsid polypeptide designed to include a binding moiety having the ability to covalently bind to the amino acid segment of another AAV can be designed to include one or more linkers and/or one or more protease cleavage sites in the various arrangements described herein.

In another example, an AAV can be designed to include a capsid polypeptide having an amino acid segment containing the amino acid sequence set forth in SEQ ID NO:1 (or a variant thereof), and a polypeptide (e.g., a conjugating polypeptide) can be designed to include a binding moiety having the ability to covalently bind to the amino acid segment of the AAV. In such a case, the AAV can be covalently bound to the polypeptide (e.g., a conjugating polypeptide) via the amino acid segment of the AAV and the binding moiety of the polypeptide (e.g., a conjugating polypeptide). Such a polypeptide (e.g., a conjugating polypeptide) containing a binding moiety having the ability to covalently bind to an amino acid segment containing the amino acid sequence set forth in SEQ ID NO:1 (or a variant thereof) that is included within a capsid polypeptide of an AAV as described herein can be used to covalently attach molecules of interest to that AAV via that polypeptide (e.g., conjugating polypeptide). For example, a conjugating polypeptide can be designed to include an enzyme (i.e., a molecule of interest in this example) and a binding moiety having the ability to covalently bind to an amino acid segment containing the amino acid sequence set forth in SEQ ID NO:1 (or a variant thereof). In this example, such a conjugating polypeptide can be used to covalently attach that enzyme to the surface of an AAV described herein that includes a capsid polypeptide having an amino acid segment containing the amino acid sequence set forth in SEQ ID NO:1 (or a variant thereof).

In some cases, a conjugating polypeptide can be designed to include two or more binding moieties having the ability to covalently bind to an amino acid segment containing the amino acid sequence set forth in SEQ ID NO:1 (or a variant thereof). In such cases, the two or more binding moieties can be identical or different. For example, a conjugating polypeptide can be designed to include a first binding moiety having the ability to covalently bind to an amino acid segment containing the amino acid sequence set forth in SEQ ID NO:1 (or a variant thereof) and a second binding moiety having the ability to covalently bind to an amino acid segment containing the amino acid sequence set forth in SEQ ID NO:1 (or a variant thereof). In some cases, a conjugating polypeptide can be designed to include a first binding moiety containing the amino acid sequence set forth in any one of SEQ ID NOs:10-17, 26, and 27 and a second binding moiety containing the amino acid sequence set forth in any one of SEQ ID NOs:10-17, 26, and 27.

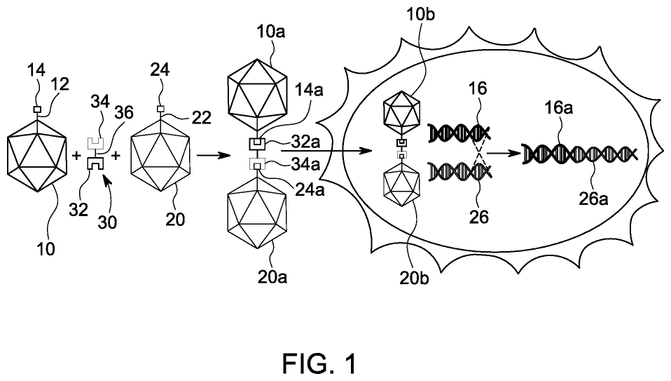

A conjugating polypeptide provided herein and designed to include two or more binding moieties having the ability to covalently bind to an amino acid segment containing the amino acid sequence set forth in SEQ ID NO:1 (or a variant thereof) can be used to covalently link two AAVs together. With reference to FIG. 1, a first AAV (10) can be designed to include a capsid polypeptide (12) containing an amino acid segment (14) containing the amino acid sequence set forth in SEQ ID NO:1 (or a variant thereof) as described herein. A second AAV (20) can be designed to include a capsid polypeptide (22) containing an amino acid segment (24) containing the amino acid sequence set forth in SEQ ID NO:1 (or a variant thereof) as described herein. In addition, a conjugating polypeptide (30) can be designed to include a first binding moiety (32) and a second binding moiety (34). In some cases, first binding moiety (32) and second binding moiety (34) can be linked with a linker sequence (36). Any appropriate linker sequence can be used to link two or more binding moieties of a conjugating polypeptide together. Examples of linker sequences that can be used to link two or more binding moieties of a conjugating polypeptide together include, without limitation, those set forth in Table 5.

TABLE 5
Example of linker sequences for
conjugating polypeptides.

| | SEQ ID |
|---|---|
| Sequence | NO: |
| SGGGGSGGGGSGGGGS | 41 |
| LAEAAAKEAAAKAAA | 42 |
| GSGGGSGGGSPANLKALEAQKQKEQRQAA | 43 |
| EELANAKKLKEQLEKGSGGGSGGGS | |

With further reference to FIG. 1, first AAV (10), second AAV (20), and conjugating polypeptide (30) can be combined to covalently link first AAV (10) to second AAV (20) via conjugating polypeptide (30). In this example, first AAV (10a) is covalently linked to second AAV (20a) via a first covalent bond between amino acid segment (14a) and first binding moiety (32a) and a second covalent bond between amino acid segment (24a) and second binding moiety (34a). In some examples, the covalently attached AAVs can enter a single cell as one unit (first AAV (10b) and second AAV (20b)), thereby delivering the nucleic acid content of the two AAVs to that cell. In some cases, first AAV (10b) and second AAV (20b) can contain different genetic material. For example, first AAV (10b) can include nucleic acid (16), and second AAV (20b) can include nucleic acid (26), which is different from that of nucleic acid (16). In some cases, the delivered nucleic from two or more covalently linked AAVs can contain overlapping sequences such that they can recombine to form a lager nucleic acid within the cell. For example, nucleic acid (16) and nucleic acid (26) can contain overlapping sequences such that they can recombine to form a lager nucleic acid shown as nucleic acid (16a) and nucleic acid (26a).

In some cases, a conjugating polypeptide provided herein can be designed to include a first binding moiety having the ability to covalently bind to an amino acid segment containing the amino acid sequence set forth in SEQ ID NO:1 (or a variant thereof) and a second binding moiety having the ability to covalently bind to an amino acid tag. In these cases, the conjugating polypeptide can be used to covalently link a first AAV that includes a capsid polypeptide having an amino acid segment containing the amino acid sequence set forth in SEQ ID NO:1 (or a variant thereof) to a second AAV that includes a capsid polypeptide having that amino acid tag. For example, with reference to FIG. 1, first AAV (10) can remain as described above, while second AAV (20) can be changed such that amino acid segment (24) is replaced with an amino acid tag. Likewise, conjugating polypeptide (30) can be changed such that second binding moiety (34) is replaced with a binding moiety having the ability to form a covalently bond with that amino acid tag.

Examples of (a) amino acid tags that can be used as described herein (e.g., for inclusion in an AAV) along with (b) their corresponding binding moieties having the ability to form a covalent bond with the amino acid tag (e.g., for inclusion in a conjugating polypeptide) include, without limitation, those set forth in Table 6.

TABLE 6
Amino acid tags and corresponding
binding moiety partners.

| | | SEQ | | SEQ |
|---|---|---|---|---|
| Ex- | Tag | ID | Binding Moiety | ID |
| ample | Sequence | NO: | Sequence | NO: |
| 1 | AHIVMVD | 8 | DSATHIKFSKRDEDGKELAG | 18 |
| | AYKPTK | | ATMELRDSSGKTISTWISDG | |
| | | | QVKDFYLYPGKYTFVETAAP | |
| | | | DGYEVATAITFTVNEQGQVT | |
| | | | VNGKATKGDAHI | |
| 2 | KLGDIEF | 9 | GSHMKPLRGAVFSLQKQHPD | 19 |
| | IKVNK | | YPDIYGAIDQNGTYQNVRTG | |
| | | | EDGKLTFKNLSDGKYRLFEN | |
| | | | SEPAGYKPVQNKPIVAFQIV | |
| | | | NGEVRDVTSIVPQDIPATYE | |
| | | | FTNGKHYITNEPIPPK | |
| 3 | VPTIVMV | 44 | AMVTTLSGLSGEQGPSGDMT | 45 |
| | DAYKRYK | | TEEDSATHIKFSKRDEDGRE | |
| | | | LAGATMELRDSSGKTISTWI | |
| | | | SDGHVKDFYLYPGKYTFVET | |
| | | | AAPDGYEVATAITFTVNEQG | |
| | | | QVTVNGEATKGDAHTGSSGS | |

AAVs having a capsid polypeptide that includes an amino acid tag as described herein can be designed using any of the approaches described herein with respect to an amino acid segment containing the amino acid sequence set forth in SEQ ID NO:1 (or a variant thereof) with the amino acid tag being used in place of the amino acid segment containing the amino acid sequence set forth in SEQ ID NO:1 (or a variant thereof). For example, an AAV having a capsid polypeptide designed to include an amino acid tag (e.g., an amino acid tag set forth in Table 6) can be designed to include one or more linkers and/or one or more protease cleavage sites in the various arrangements described herein.

This document also provides compositions containing an AAV described herein. For example, compositions can be produced to contain one or more AAVs having a capsid polypeptide that includes an amino acid segment containing the amino acid sequence set forth in SEQ ID NO:1 (or a variant thereof) as described herein. In some cases, such a composition can include the AAVs at a titer that is greater than 10¹⁴ (e.g., greater than 1×10¹⁴, greater than 2×10¹⁴, greater than 3×10¹⁴, greater than 4×10¹⁴, greater than 5×10¹⁴, greater than 6×10¹⁴, greater than 7×10¹⁴, greater than 8×10¹⁴, or greater than 9×10¹⁴). In some cases, a composition provided herein can have AAVs with a titer that is greater than (e.g., at least 5-fold, 10-fold, 25-fold, 50-fold, 75-fold, or 100-fold greater than) the titer of an AAV preparation of a comparable AAV that lacks an amino acid segment containing the amino acid sequence set forth in SEQ ID NO:1 (or a variant thereof). For example, a composition provided herein having AAV designed to contain a capsid polypeptide that includes an amino acid segment containing the amino acid sequence set forth in SEQ ID NO:1 (or a variant thereof) can have an AAV titer that is greater than (e.g., at least 5-fold, 10-fold, 25-fold, 50-fold, 75-fold, or 100-fold greater than) an AAV preparation of a comparable AAV that contains an amino acid tag having the amino acid sequence set forth in SEQ ID NO:8 or SEQ ID NO:9, instead of SEQ ID NO:1 (or a variant thereof).

This document also provides nucleic acid molecules encoding an AAV described herein. For example, isolated nucleic acid molecules provided herein can be designed to encode one or more AAVs having a capsid polypeptide that includes an amino acid segment containing the amino acid sequence set forth in SEQ ID NO:1 (or a variant thereof) described herein. In some cases, a nucleic acid molecule provided herein can encode one or more AAVs having a capsid polypeptide that includes one or more linkers (e.g., any of the exemplary linkers described herein) and an amino acid segment containing the amino acid sequence set forth in SEQ ID NO:1 (or a variant thereof). For example, a nucleic acid molecule can be designed to encode an AAV having a capsid polypeptide that includes an N-terminal linker that includes a threonine and glycine (TG) linker sequence immediately preceding the amino acid segment and a C-terminal linker that includes a glycine, leucine, and serine (GLS) sequence immediately following the amino acid segment. In another example, a nucleic acid molecule provided herein can encode for an AAV having a capsid polypeptide that includes an N-terminal linker that includes a leucine and an alanine (LA) linker sequence immediately preceding the amino acid segment and a C-terminal linker that includes an alanine (A) immediately following the amino acid segment. In some cases, nucleic acid molecules provided herein can encode one or more AAVs having a capsid polypeptide that includes a protease cleavage site (e.g., any of the exemplary protease cleavage sites described herein) and an amino acid segment containing the amino acid sequence set forth in SEQ ID NO:1 (or a variant thereof) as described herein. For example, a nucleic acid molecule provided herein can encode an AAV having a capsid polypeptide that includes an N-terminal protease cleavage site immediately preceding the amino acid segment. In another example, a nucleic acid molecule provided herein can encode an AAV having a capsid polypeptide that includes an N-terminal linker immediately preceding a protease cleavage site that is N-terminal to an amino acid segment set forth in SEQ ID NO:1 (or a variant thereof) as described herein.

In some cases, this document provides nucleic acid molecules that can encode one or more AAVs having a capsid polypeptide that includes an amino acid segment as set forth in SEQ ID NO:1 located at amino acid position 588 (or an amino acid position within three amino acids of position 588). In some cases, this document provides nucleic acid molecules that can encode one or more AAVs having a capsid polypeptide that includes an amino acid segment as set forth in SEQ ID NO:1 located at amino acid position 453 (or at an amino acid position within three amino acids of position 453). In some cases, this document provides nucleic acid molecules that can encode one or more AAVs having a capsid polypeptide that includes an amino acid tag (e.g., any of the exemplary amino acid tags described herein) located at amino acid position 588 (e.g., or at an amino acid position within three amino acids of position 588). In some cases, this document provides nucleic acid molecules that can encode one or more AAVs having a capsid polypeptide that includes an amino acid tag (e.g., any of the exemplary amino acid tags described herein) located at amino acid position 453 (or an amino acid position within three amino acids of position 453).

In some cases, this document provides nucleic acid molecules that can encode one or more AAVs having a capsid polypeptide that includes a binding moiety that can covalently bind to the amino acid segment set forth in SEQ ID NO:1 (or a variant thereof). For example, nucleic acid molecules provided herein can encode one or more AAVs having a capsid polypeptide that includes a binding moiety having the ability to form a covalent bond with an amino acid segment containing the amino acid sequence set forth in SEQ ID NO:1 (or a variant thereof). This document also provides conjugating polypeptides (and nucleic acids encoding conjugating polypeptides). For example, conjugating polypeptides provided herein can be designed to contain one or more binding moieties having the ability to bind covalently to the amino acid segment set forth in SEQ ID NO:1 (or a variant thereof). In some cases, conjugating polypeptides are provided herein that include a binding moiety having the ability to form a covalent bond with an amino acid segment containing the amino acid sequence set forth in SEQ ID NO:1 (or a variant thereof) can have the amino acid sequence as set forth in any one of SEQ ID NOs:10-17, 26, and 27.

In some cases, a conjugating polypeptide provided herein can include two or more binding moieties where at least one binding moiety includes the ability to bind covalently to the amino acid segment set forth in SEQ ID NO:1 (or a variant thereof). For example, a conjugating polypeptides provided herein can include a first binding moiety having the ability to bind covalently to the amino acid segment set forth in SEQ ID NO:1 (or a variant thereof) and a second binding moiety having the ability to bind covalently to the amino acid segment set forth in SEQ ID NO:1 (or a variant thereof). In another example, a conjugating polypeptide can include a first binding moiety having the ability to bind covalently to the amino acid segment set forth in SEQ ID NO:1 (or a variant thereof) and a second binding moiety having the ability to covalently bind to an amino acid tag (e.g., any of the exemplary amino acid tags provided herein). In some cases, a conjugating polypeptides provided herein can include a linker (e.g., any of the exemplary linkers described herein) located between the two binding moieties. In some cases, the two or more binding moieties directly abut each other in the conjugating polypeptide.

Also provided in this document are nucleic acid molecules encoding any of the exemplary conjugating polypeptides described herein. For example, isolated nucleic acid molecules provided herein can encode for a conjugating polypeptide that includes a binding moiety (e.g., any of the exemplary binding motifs described herein) that binds covalently to the amino acid segment present in the capsid polypeptide of an AAV. In such cases, a nucleic acid molecule provided herein can encoding the amino acid sequence set forth in SEQ ID NOs:10-17, 26, and 27.

In addition, this document provides methods for making a composition that includes two or more different AAVs covalently linked together. For example, compositions can be produced to contain two or more AAVs linked together through a covalent bond. In such cases, the covalent bond can be provided by an interaction between a first AAV having a capsid polypeptide that includes an amino acid segment containing the amino acid sequence set forth in SEQ ID NO:1 (or a variant thereof) as described herein and a second AAV having a capsid polypeptide that includes a binding moiety (e.g., any of the exemplary binding motifs described herein) that binds covalently to the amino acid segment present in the capsid polypeptide of an AAV. For example, a second AAV can include a capsid polypeptide that includes a binding moiety having the amino acid sequence set forth in SEQ ID NOs:10-17, 26, and 27.

In some cases, compositions provided herein can include two or more different AAVs covalently linked together through each AAV's covalent bond with a conjugating polypeptide (e.g., any of the exemplary conjugating polypeptides described herein). For example, a conjugating polypeptide of any one of the compositions provided herein can include at least one binding moiety having the ability to form a covalent bond with an amino acid segment containing the amino acid sequence set forth in SEQ ID NO:1 (or a variant thereof). In such cases, the binding moiety having the ability to form a covalent bond with the amino acid segment of SEQ ID NO:1 can have the amino acid sequence as set forth in any one of the sequences set forth in SEQ ID NOs:10-17, 26, and 27.

In some cases, a conjugating polypeptide of any one of the compositions described herein can include two or more binding moieties. In such cases, the amino acid sequences of the two or more binding moieties can be identical or different. In some cases, a conjugating polypeptide of any one of the compositions provided herein can include at least one binding moiety having the ability to form a covalent bond with an amino acid tag (e.g., any of the exemplary amino acid tags described herein).

In some cases, this document provides methods for generating a composition including a first AAV covalently linked to a second AAV. Such methods can include providing (i) a first AAV including a capsid polypeptide having an amino acid segment containing the amino acid sequence set forth in SEQ ID NO:1 (or a variant thereof) and (ii) a second AAV including a capsid polypeptide containing an amino acid tag. Such a method can then include contacting the first AAV with a conjugating polypeptide to form a first AAV-conjugating polypeptide complex. Such a conjugating polypeptide can be designed to include (i) a first binding moiety having the ability to bind covalently to the amino acid segment of the first AAV and (ii) a second binding moiety having the ability to bind covalently to the amino acid tag of a second AAV. In such cases, the first AAV-conjugating polypeptide complex can be contacted with the second AAV to form a “first AAV-conjugating polypeptide-second AAV” complex.

In some cases, this document provides methods for generating a composition including a first AAV covalently linked to a second AAV. Such a method can include providing (i) a first AAV that includes a capsid polypeptide containing an amino acid segment including the amino acid sequence set forth in SEQ ID NO:1 (or a variant thereof) and (ii) a second AAV that includes a capsid polypeptide containing an amino acid tag. Such a method can then include contacting the second AAV with a conjugating polypeptide to form a second AAV-conjugating polypeptide complex. In such cases, the conjugating polypeptide can include (i) a first binding motif having the ability to bind covalently to the amino acid segment of the first AAV and (ii) a second binding motif having the ability to bind covalently to the amino acid tag of the second AAV. In such cases, a complex of the second AAV-conjugating polypeptide can be contacted to the first AAV to form a “second AAV-conjugating polypeptide-first AAV” complex.

In some cases, a composition provided herein can be purified after the formation of a first AAV-conjugating polypeptide complex or after the formation of a second AAV-conjugating polypeptide complex. In such cases, the purification can enrich a solution for an AAV-conjugating polypeptide complex. In some cases, a composition provided herein can undergo a purification step after formation of the “first AAV-conjugating polypeptide-second AAV” complex or after formation of the “second AAV-conjugating polypeptide-first AAV” complex. In such cases, the purification result in a composition enriched for the AAV-conjugating polypeptide-AAV complexes.

The invention will be further described in the following examples, which do not limit the scope of the invention described in the claims.

### EXAMPLES

### Example 1—Construction of AAV Vector Including an Amino Acid Segment in a VP3 Polypeptide

### Construction of AAV Vector Including Amino Acid Segment and LA and a Linkers

An AAV2 vector was generated that includes an amino acid sequence set forth in SEQ ID NO:1 inserted at amino acid position 588 of the VP3 polypeptide. The AAV2 vector was designed to encode an N-terminal linker of leucine and alanine (LA) immediately preceding the amino acid segment. The AAV2 vector also was designed to encode a C-terminal linker of alanine (A) immediately following the amino acid segment. The amino acid segment with the LA and A linkers was referred to as the “LA-AAS-A polypeptide.”

An empty AAV2 vector was amplified with a forward primer having a sequence of 5′-AGACAAGCAGCTACCGCAGAT-3′ (SEQ ID NO:28) and a reverse primer having a sequence of 5′-GTTGCCTCTCTGGAGGTTGG-3′ (SEQ ID NO:29). Gibson assembly (New England Biolabs) was used to anneal the nucleic acid sequence encoding the LA-AAS-A polypeptide into the PCR amplified nucleic acid of the AAV such that it is located at amino acid position 588 of the VP3 polypeptide, resulting nucleic acid encoding AAV2-588-LA-AAS-A. The final vector included a green fluorescent protein (GFP) to monitor virus transduction.

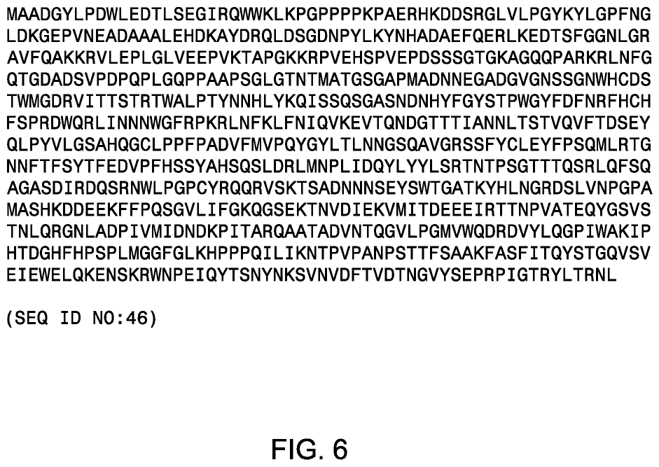

The nucleic acid sequence encoding the LA-AAS-A polypeptide included the sequence of: CTAGCAGACCCTATTGTAATGATTGATAATGACAAGC-CGATTACTGCT (SEQ ID NO:30). The amino acid sequence of AAV2-588-LA-AAS-A included the amino acid sequence set forth in FIG. 6.

### Construction of AAV Vector Including an Amino Acid Segment and TG and GLS Linkers

An AAV2 vector was generated that includes an amino acid segment set forth in SEQ ID NO:1 inserted at amino acid position 588 of the VP3 polypeptide. The AAV2 vector included an N-terminal linker of threonine and glycine (TG) immediately preceding the amino acid segment. The AAV2 vector also included a C-terminal linker of glycine, leucine, and serine (GLS) immediately following the amino acid segment. The amino acid segment with the TG and GLS linkers was referred to as “TG-AAS-GLS polypeptide.”

An empty AAV2 vector was amplified with a forward primer having a sequence of 5′-AGACAAGCAGCTACCGCAGAT-3′ (SEQ ID NO:31) and a reverse primer having a sequence of 5′-GTTGCCTCTCTGGAGGTTGG-3′ (SEQ ID NO:32). Gibson assembly (New England Biolabs) was used to anneal the nucleic acid sequence encoding the TG-AAS-GLS polypeptide into the PCR amplified nucleic acid of the AAV2 such that it is located at amino acid position 588 of the VP3 polypeptide, resulting nucleic acid encoding AAV2-588-TG-AAS-GLS. The final vector included GFP to monitor virus transduction.

The nucleic acid sequence encoding the TG-AAS-GLS polypeptide included the sequence of: ACCGGTGACCCTATTGTAATGATTGATAATGACAA-GCCGATTACTGGCTTAAGT (SEQ ID NO:33).

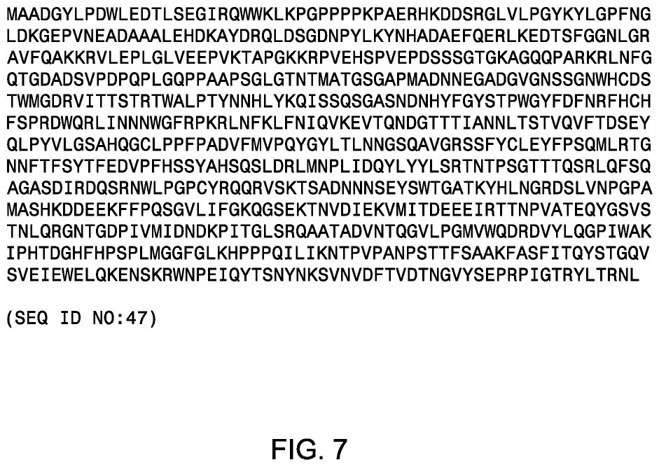

The amino acid sequence of AAV2-588-TG-AAS-GLS included the amino acid sequence set forth in FIG. 7.

AAV vectors containing SpyTag or SnoopTag were constructed as described elsewhere (International Patent Application No. PCT/US2019/020096)

### Production of AAV and Assessment of AAV Titer

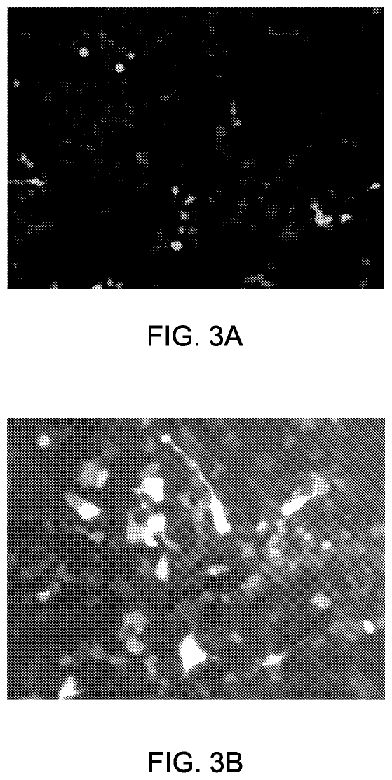

The AAV vectors were used to make viruses. Briefly, each AAV vector was transfected according to the methods described elsewhere (Grieger et al., Nat. Protoc., 1(3):1412-28 (2006)). Recombinant AAV was purified by iodixanol gradient ultracentrifugation followed by a buffer exchange and concentration with Amicon Ultra-15 Centrifugal Filter Units in PBS+0.001% Pluronic F-68. Titers were determined by quantitative PCR relative to a standard curve (Aurnhammer et al., Hum. Gene Ther. Methods., 23(1):18-28 (2012)) in a manner that allowed the titers to be compared. Table 7 shows that AAVs including a capsid polypeptide having the amino acid segment containing SEQ ID NO:1 produced 10-fold to 100-fold greater titers than AAVs including a capsid polypeptide having the amino acid segment containing either SpyTag or SnoopTag. FIGS. 3A and 3B show GFP expression following in vitro infection of 5.0×10⁵ 293 cells with 1 μL of AAV2-588-TG-SpyTag-GLS (FIG. 3A) or 1 μL AAV2-588-TG-AAS-GLS. Comparing FIG. 3A (AVV-TG-SpyTag-GLS) with FIG. 3B (AAV2-TG-AAS-GLS) confirms the higher titer of the AAV2-TG-AAS-GLS virus.

TABLE 7
Titer results.

| Backbone | Inserted Sequence | Linkers | Titer |
|---|---|---|---|
| AAV2 | SEQ ID NO: 1 | TG . . . GLS | 5.5 × 10¹⁴ |
| AAV2 | SEQ ID NO: 1 | TG . . . GLS | 9.0 × 10¹⁴ |
| AAV2 | SEQ ID NO: 1 | TG . . . GLS | 8.9 × 10¹⁴ |
| AAV2 | SEQ ID NO: 1 | TG . . . GLS | 4.6 × 10¹⁴ |
| AAV2 | SnoopTag | TG . . . GLS | 1.78 × 10¹³ |
| AAV2 | SnoopTag | TG . . . GLS | 2.7 × 10¹² |
| AAV2 | SnoopTag | TG . . . GLS | 2.8 × 10¹³ |
| AAV2 | SpyTag | TG . . . GLS | 3.3 × 10¹² |
| AAV2 | SpyTag | TG . . . GLS | 5.5 × 10¹² |

These results demonstrate that AAVs containing a capsid polypeptide including an amino acid segment containing SEQ ID NO:1 (or a variant thereof) are significantly superior to comparable AAVs with capsid polypeptides including an inserted sequence that is not SEQ ID NO:1 (or a variant thereof).

### Example 2—Protease-Mediated Cleavage to Release Amino Acid Segments and Enhance Binding to a Binding Moiety

An AAV system was generated that incorporates a tev protease cleavage site within 30 amino acid residues of an amino acid segment containing the sequence set forth in SEQ ID NO:1 (or a variant thereof) or a tev protease cleavage site within 30 amino acid residues of an amino acid tag such as SpyTag or SnoopTag. As shown in FIG. 4, an AAV was designed to allow for tev protease-mediated cleavage of a tev protease cleavage site up stream (or downstream) of an amino acid segment. Following cleavage, one end of the amino acid segment (e.g., either the 5′ or 3′ end depending on the position of the tev protease cleavage site relative to the amino acid segment) is to be released from at least a portion of the capsid polypeptide. This cleavage (on either a 5′ or 3′ end) is designed to enhance binding of the amino acid segment to a binding moiety.

To confirm successful use, the following set of experiments was performed. In these experiments, AAV2 vectors including a tev protease cleavage site immediately preceding a SpyTag at amino acid position 588 of the VP3 polypeptide were constructed, the AAV2 viruses were produced, and the infectivity of the AAV2 viruses was tested.

### Construction of AAV Vectors

A set of AAV vectors was generated where a SpyTag was inserted at amino acid position 588 of the VP3 polypeptide. A first AAV2 vector included nucleic acid encoding an N-terminal linker of leucine and alanine (LA) immediately preceding the tev protease cleavage site and a C-terminal linker of alanine (A) immediately following the SpyTag (“LA-tev-SpyTag-A polypeptide”). A second AAV2 vector included nucleic acid encoding an N-terminal linker of threonine and glycine (TG) immediately preceding the tev protease cleavage site and a C-terminal linker of glycine, leucine, and serine (GLS) immediately following the SpyTag (“TG-tev-SpyTag-GLS polypeptide”).

The nucleic acid sequence encoding the LA-tev-SpyTag-A polypeptide was as follows: CTAGCAGAGAACCTCTATTTCCAGGGAGCCCACATCGTGAT-GGTGGACGCCTACAAGCCGACGAAGGCT (SEQ ID NO:34).

The nucleic acid sequence encoding the TG-tev-SpyTag-GLS polypeptide was as follows: ACCGGTGAGAACCTCTATTTCCAGGGAGCCCACATCGTGA-TGGTGGACGCCTACAAGCCGACGAAGGGCTTAAGT (SEQ ID NO:35).

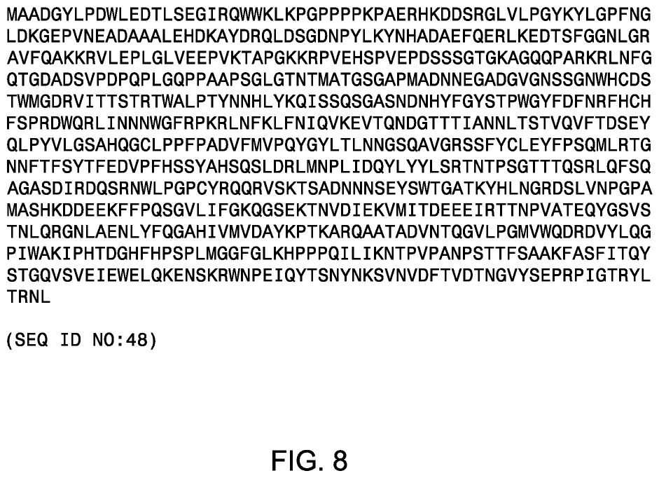

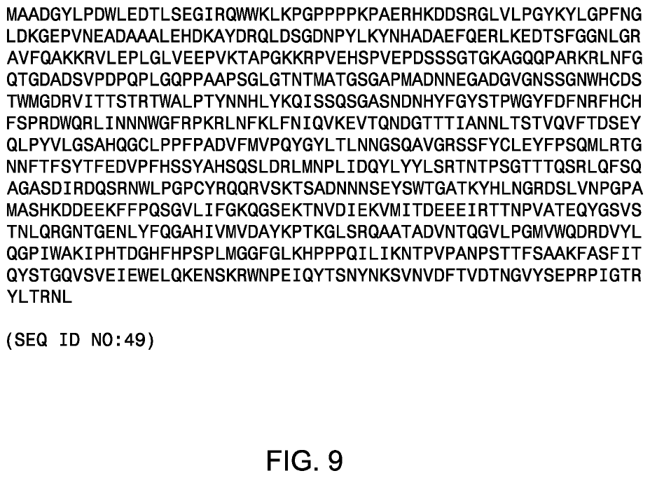

Gibson Assembly (New England Biolabs) was used to assemble the nucleic acid sequence encoding the LA-tev-SpyTag-A polypeptide or TG-tev-SpyTag-GLS polypeptide into the PCR amplified nucleic acid of the AAV2 constructs such that it is located at amino acid position 588 of the VP3 polypeptide, resulting nucleic acid encoding AAV2-588-LA-tev-SpyTag-A or AAV2-588-TG-tev-SpyTag-GLS, respectively. The amino acid sequence of AAV2-588-LA-tev-SpyTag-A included the amino acid sequence set forth in FIG. 8, and the amino acid sequence encoding AAV2-588-TG-tev-SpyTag-GLS included the amino acid sequence set forth in FIG. 9.

### Production and Assessment of AAVs

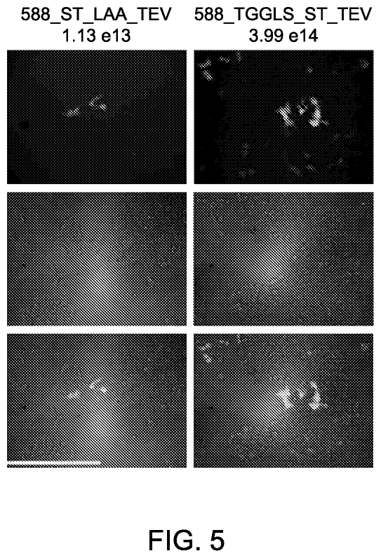

The AAV2 vectors were used to make virus according to the methods described herein. FIG. 5 lists the viral titers of each virus and demonstrates GFP expression 48 hours after in vitro infection of HEK 293 cells with 10 μL of virus. GFP expression revealed that the tev protease cleavage site and protease cleavage did not interfere with successful virus production or expression of nucleic acid delivered to cells by AAVs.

### Example 3—Preparation of a Composition Containing Two AAVs Covalently Attached to Each Other

A composition including a first AAV (e.g., an AAV2-TG-AAS-GLS virus or an AAV2-TG-Tev-AAS-GLS virus) and a second AAV (e.g., an AAV2-TG-SpyTag-GLS virus or an AAV2-TG-Tev-SpyTag-GLS virus) covalently bound together by a conjugating polypeptide that includes a first binding moiety that covalently binds to the amino acid segment of the first AAV virus and a second binding moiety that covalently binds to the SpyTag of the second AAV is produced. The first AAV is designed to include nucleic acid encoding a GFP fluorescent protein as a marker for transduction, and the second AAV is designed to include nucleic acid encoding an mCherry fluorescent protein as a marker for transduction.

A preparation of the first AAV is produced to have a titer greater than 10¹⁴, while a preparation of the second AAV is produced to have a titer greater than 10¹². A conjugating polypeptide is produced to include a first binding moiety that covalently binds to the amino acid segment of the first AAV virus and a second binding moiety that covalently binds to the SpyTag of the second AAV.

In one example, the preparation of the first AAV is optionally treated with a protease (e.g., a Tev protease), and the resulting preparation is mixed with the conjugating polypeptide under conditions wherein the first binding moiety of the conjugating polypeptide covalently binds to the amino acid segment of the first AAV to form a first AAV-conjugation polypeptide complex. The mixture ratio is 10:1, 5:1, 2:1, 1:1, 1:2, 1:5, and 1:10 of first AAV to conjugating polypeptide. Then, the first AAV-conjugation polypeptide complex is optionally purified to produce an enriched preparation of the first AAV-conjugation polypeptide complex, and the first AAV-conjugation polypeptide complex (or enriched preparation) is mixed with the second AAV under conditions wherein the second binding moiety of the conjugating polypeptide covalently binds to the amino acid tag of the second AAV to form a first AAV-conjugation polypeptide-second AAV complex. The mixture ratio is 10:1, 5:1, 2:1, 1:1, 1:2, 1:5, and 1:10 of complex to second AAV. The first AAV-conjugation polypeptide-second AAV complex is optionally purified to produce an enriched preparation of the first AAV-conjugation polypeptide-second AAV complex. The first AAV-conjugation polypeptide-second AAV complex is used to transfect cells, and expression of GFP and mCherry within the same cell is used to confirm delivery of the covalently linked AAVs to the same cell.

In another example, the preparation of the second AAV is mixed with the conjugating polypeptide under conditions wherein the second binding moiety of the conjugating polypeptide covalently binds to the amino acid tag of the second AAV to form a second AAV-conjugation polypeptide complex. The mixture ratio is 10:1, 5:1, 2:1, 1:1, 1:2, 1:5, and 1:10 of second AAV to conjugating polypeptide. Then, the second AAV-conjugation polypeptide complex is optionally purified to produce an enriched preparation of the second AAV-conjugation polypeptide complex, and the second AAV-conjugation polypeptide complex is mixed with the first AAV under conditions wherein the first binding moiety of the conjugating polypeptide covalently binds to the amino acid segment of the first AAV to form a second AAV-conjugation polypeptide-first AAV complex. The mixture ratio is 10:1, 5:1, 2:1, 1:1, 1:2, 1:5, and 1:10 of complex to first AAV. The second AAV-conjugation polypeptide-first AAV complex is optionally purified to produce an enriched preparation of the second AAV-conjugation polypeptide-first AAV complex. The second AAV-conjugation polypeptide-first AAV complex is used to transfect cells, and expression of GFP and mCherry within the same cell is used to confirm delivery of the covalently linked AAVs to the same cell.

### Example 4—Additional Embodiments

Embodiment 1. An Adeno-Associated Virus (AAV) comprising a capsid polypeptide, wherein said capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions.
Embodiment 2. The AAV of embodiment 1, wherein said AAV is an AAV2.
Embodiment 3. The AAV of any one of embodiments 1-2, wherein said amino acid segment comprises the amino acid sequence set forth in SEQ ID NO:1 with three amino acid substitutions.
Embodiment 4. The AAV of any one of embodiments 1-2, wherein said amino acid segment comprises the amino acid sequence set forth in SEQ ID NO:1 with two amino acid substitutions.
Embodiment 5. The AAV of any one of embodiments 1-2, wherein said amino acid segment comprises the amino acid sequence set forth in SEQ ID NO:1 with one amino acid substitutions.
Embodiment 6. The AAV of any one of embodiments 1-2, wherein said amino acid segment comprises the amino acid sequence set forth in SEQ ID NO:1.
Embodiment 7. The AAV of any one of embodiments 1-6, wherein said capsid polypeptide comprises said amino acid segment at an amino acid position within three amino acid residues of an amino acid residue corresponding to position 453 or position 588 of a reference VP1 polypeptide having the amino acid sequence set forth in SEQ ID NO:2.
Embodiment 8. The AAV of any one of embodiments 1-6, wherein said capsid polypeptide comprises said amino acid segment at an amino acid position within three amino acid residues of an amino acid residue corresponding to position 453 of a reference VP1 polypeptide having the amino acid sequence set forth in SEQ ID NO:2.
Embodiment 9. The AAV of any one of embodiments 1-6, wherein said capsid polypeptide comprises said amino acid segment at an amino acid position within two amino acid residues of an amino acid residue corresponding to position 453 of a reference VP1 polypeptide having the amino acid sequence set forth in SEQ ID NO:2.
Embodiment 10. The AAV of any one of embodiments 1-6, wherein said capsid polypeptide comprises said amino acid segment at an amino acid position within one amino acid residue of an amino acid residue corresponding to position 453 of a reference VP1 polypeptide having the amino acid sequence set forth in SEQ ID NO:2.
Embodiment 11. The AAV of any one of embodiments 1-6, wherein said capsid polypeptide comprises said amino acid segment at an amino acid position of an amino acid residue corresponding to position 453 of a reference VP1 polypeptide having the amino acid sequence set forth in SEQ ID NO:2.
Embodiment 12. The AAV of any one of embodiments 1-6, wherein said capsid polypeptide comprises said amino acid segment at an amino acid position within three amino acid residues of an amino acid residue corresponding to position 588 of a reference VP1 polypeptide having the amino acid sequence set forth in SEQ ID NO:2.
Embodiment 13. The AAV of any one of embodiments 1-6, wherein said capsid polypeptide comprises said amino acid segment at an amino acid position within two amino acid residues of an amino acid residue corresponding to position 588 of a reference VP1 polypeptide having the amino acid sequence set forth in SEQ ID NO:2.
Embodiment 14. The AAV of any one of embodiments 1-6, wherein said capsid polypeptide comprises said amino acid segment at an amino acid position within one amino acid residue of an amino acid residue corresponding to position 588 of a reference VP1 polypeptide having the amino acid sequence set forth in SEQ ID NO:2.
Embodiment 15. The AAV of any one of embodiments 1-6, wherein said capsid polypeptide comprises said amino acid segment at an amino acid position of an amino acid residue corresponding to position 588 of a reference VP1 polypeptide having the amino acid sequence set forth in SEQ ID NO:2.
Embodiment 16. The AAV of any one of embodiments 1-15, wherein said capsid polypeptide comprises an N-terminal linker immediately preceding said amino acid segment or preceding said amino acid segment by no more than ten amino acid residues.
Embodiment 17. The AAV of embodiment 16, wherein said N-terminal linker comprises (a) a leucine and alanine (LA) sequence or (b) a threonine and glycine (TG) sequence.
Embodiment 18. The AAV of any one of embodiments 1-17, wherein said capsid polypeptide comprises a C-terminal linker immediately following said amino acid segment or following said amino acid segment by no more than ten amino acid residues.
Embodiment 19. The AAV of embodiment 18, wherein said C-terminal linker comprises (a) an alanine (A) or (b) a glycine, leucine, and serine (GLS) sequence.
Embodiment 20. The AAV of any one of embodiments 1-15, wherein said capsid polypeptide comprises a leucine and alanine (LA) sequence immediately preceding said amino acid segment, and wherein said capsid polypeptide comprises an alanine immediately following said amino acid segment.
Embodiment 21. The AAV of any one of embodiments 1-15, wherein said capsid polypeptide comprises a threonine and glycine (TG) sequence immediately preceding said amino acid segment, and wherein said capsid polypeptide comprises a glycine, leucine, and serine (GLS) sequence immediately following said amino acid segment.
Embodiment 22. The AAV of any one of embodiments 1-21, wherein said capsid polypeptide comprises a protease cleavage site.
Embodiment 23. The AAV of embodiment 22, wherein said protease cleavage site is a cleavage site for a protease selected from the group consisting of a TEV protease, a PreScission protease, thrombin, Factor Xa, and enterokinase.
Embodiment 24. The AAV of any one of embodiments 22-23, wherein said protease cleavage site comprises the amino acid sequence set forth in any one of SEQ ID NOs:20-25.
Embodiment 25. The AAV of any one of embodiments 22-24, wherein said protease cleavage site immediately precedes said amino acid segment or precedes said amino acid segment by no more than 20 amino acid residues.
Embodiment 26. The AAV of any one of embodiments 22-24, wherein said protease cleavage site precedes said amino acid segment by no more than ten amino acid residues.
Embodiment 27. The AAV of any one of embodiments 22-24, wherein said protease cleavage site immediately follows said amino acid segment or follows said amino acid segment by no more than 20 amino acid residues.
Embodiment 28. The AAV of any one of embodiments 22-24, wherein said protease cleavage site follows said amino acid segment by no more than ten amino acid residues.
Embodiment 29. The AAV of any one of embodiments 1-15, wherein said capsid polypeptide comprises an N-terminal linker immediately preceding a protease cleavage site or preceding said protease cleavage site by no more than ten amino acid residues, wherein said protease cleavage site immediately precedes said amino acid segment or precedes said amino acid segment by no more than ten amino acid residues.
Embodiment 30. The AAV of embodiment 29, wherein said N-terminal linker comprises (a) a leucine and alanine (LA) sequence or (b) a threonine and glycine (TG) sequence.
Embodiment 31. The AAV of any one of embodiments 29-30, wherein said capsid polypeptide comprises a C-terminal linker immediately following said amino acid segment or following said amino acid segment by no more than ten amino acid residues.
Embodiment 32. The AAV of embodiment 31, wherein said C-terminal linker comprises (a) an alanine (A) or (b) a glycine, leucine, and serine (GLS) sequence.
Embodiment 33. The AAV of any one of embodiments 29-32, wherein said capsid polypeptide comprises a leucine and alanine (LA) sequence immediately preceding said protease cleavage site, wherein said protease cleavage site immediately precedes said amino acid segment, and wherein an alanine immediately follows said amino acid segment.
Embodiment 34. The AAV of any one of embodiments 29-32, wherein said capsid polypeptide comprises a threonine and glycine (TG) sequence immediately preceding said protease cleavage site, wherein said protease cleavage site immediately precedes said amino acid segment, and wherein a glycine, leucine, and serine (GLS) sequence immediately follows said amino acid segment.
Embodiment 35. The AAV of any one of embodiments 1-15, wherein said capsid polypeptide comprises an N-terminal linker immediately preceding said amino acid segment or preceding said amino acid segment by no more than ten amino acid residues, and wherein said capsid polypeptide comprises a protease cleavage site immediately following said amino acid segment or following said amino acid segment by no more than ten amino acid residues.
Embodiment 36. The AAV of embodiment 35, wherein said N-terminal linker comprises (a) a leucine and alanine (LA) sequence or (b) a threonine and glycine (TG) sequence.
Embodiment 37. The AAV of any one of embodiments 35-36, wherein said capsid polypeptide comprises a C-terminal linker immediately following said protease cleavage site or following said protease cleavage site by no more than ten amino acid residues.
Embodiment 38. The AAV of embodiment 37, wherein said C-terminal linker comprises (a) an alanine (A) or (b) a glycine, leucine, and serine (GLS) sequence.
Embodiment 39. The AAV of any one of embodiments 35-38, wherein said capsid polypeptide comprises a leucine and alanine (LA) sequence immediately preceding said amino acid segment, wherein said protease cleavage site immediately follows said amino acid segment, and wherein an alanine immediately follows said protease cleavage site.
Embodiment 40. The AAV of any one of embodiments 35-38, wherein said capsid polypeptide comprises a threonine and glycine (TG) sequence immediately preceding said amino acid segment, wherein said protease cleavage site immediately follows said amino acid segment, and wherein a glycine, leucine, and serine (GLS) sequence immediately follows said protease cleavage site.
Embodiment 41. The AAV of any one of embodiments 1-15, wherein said capsid polypeptide comprises a C-terminal linker immediately following a protease cleavage site or following said protease cleavage site by no more than ten amino acid residues, and wherein said protease cleavage site immediately follows said amino acid segment or follows said amino acid segment by no more than ten amino acid residues.
Embodiment 42. The AAV of embodiment 41, wherein said C-terminal linker comprises (a) an alanine (A) or (b) a glycine, leucine, and serine (GLS) sequence.
Embodiment 43. The AAV of any one of embodiments 41-42, wherein said capsid polypeptide comprises an N-terminal linker immediately preceding said amino acid segment or preceding said amino acid segment by no more than ten amino acid residues.
Embodiment 44. The AAV of embodiment 43, wherein said N-terminal linker comprises (a) a leucine and alanine (LA) sequence or (b) a threonine and glycine (TG) sequence.
Embodiment 45. The AAV of any one of embodiments 29-44, wherein said protease cleavage site is a cleavage site for a protease selected from the group consisting of a TEV protease, a PreScission protease, thrombin, Factor Xa, and enterokinase.
Embodiment 46. The AAV of any one of embodiments 29-45, wherein said protease cleavage site comprises the amino acid sequence set forth in any one of SEQ ID NOs:20-25.
Embodiment 47. A composition comprising an Adeno-Associated Virus (AAV) of any one of embodiments 1-46.
Embodiment 48. The composition of embodiment 47, wherein the titer of said AAV of said composition is greater than 10¹⁴.
Embodiment 49. An isolated nucleic acid encoding an Adeno-Associated Virus (AAV) of any one of embodiments 1-46.
Embodiment 50. A composition comprising a first Adeno-Associated Virus (AAV) and a second AAV genetically different from said first AAV, wherein said first AAV is an AAV of any one of embodiments 1-46.
Embodiment 51. The composition of embodiment 50, wherein said first and second AAVs are covalently attached to each other.
Embodiment 52. The composition of embodiment 51, wherein said first AAV is covalently attached to said second AAV via at least one covalent bond of said amino acid segment of said first AAV.
Embodiment 53. The composition of any one of embodiments 50-52, wherein the capsid polypeptides of said first and second AAVs are identical.
Embodiment 54. The composition of any one of embodiments 50-52, wherein the capsid polypeptides of said first and second AAVs are different.
Embodiment 55. The composition of embodiment 54, wherein the capsid polypeptides of said second AAV lacks said amino acid segment.
Embodiment 56. The composition of any one of embodiments 50-54, wherein said second AAV comprises a capsid polypeptide comprising an amino acid tag comprising:

(a) the amino acid sequence set forth in SEQ ID NO:1 or the amino acid sequence set forth in SEQ ID NO:1 with one, two, or three amino acid substitutions,

(b) the amino acid sequence set forth in SEQ ID NO:8 or the amino acid sequence set forth in SEQ ID NO:8 with one, two, or three amino acid substitutions,

(c) the amino acid sequence set forth in SEQ ID NO:9 or the amino acid sequence set forth in SEQ ID NO:9 with one, two, or three amino acid substitutions, or

(d) the amino acid sequence set forth in SEQ ID NO:44 or the amino acid sequence set forth in SEQ ID NO:44 with one, two, or three amino acid substitutions.

Embodiment 57. The composition of embodiment 56, wherein said capsid polypeptide of said second AAV comprises said amino acid tag at an amino acid position within three amino acid residues of an amino acid residue corresponding to position 453 or position 588 of a reference VP1 polypeptide having the amino acid sequence set forth in SEQ ID NO:2.
Embodiment 58. The composition of any one of embodiments 56-57, wherein said capsid polypeptide of said second AAV comprises an N-terminal linker immediately preceding said amino acid tag or preceding said amino acid tag by no more than ten amino acid residues.
Embodiment 59. The composition of embodiment 58, wherein said N-terminal linker comprises (a) a leucine and alanine (LA) sequence or (b) a threonine and glycine (TG) sequence.
Embodiment 60. The composition of any one of embodiments 56-59, wherein said capsid polypeptide of said second AAV comprises a C-terminal linker immediately following said amino acid tag or following said amino acid tag by no more than ten amino acid residues.
Embodiment 61. The composition of embodiment 60, wherein said C-terminal linker comprises (a) an alanine (A) or (b) a glycine, leucine, and serine (GLS) sequence.
Embodiment 62. The composition of any one of embodiments 56-61, wherein said capsid polypeptide of said second AAV comprises a leucine and alanine (LA) sequence immediately preceding said amino acid tag, and wherein said capsid polypeptide of said second AAV comprises an alanine immediately following said amino acid tag.
Embodiment 63. The composition of any one of embodiments 56-61, wherein said capsid polypeptide of said second AAV comprises a threonine and glycine (TG) sequence immediately preceding said amino acid tag, and wherein said capsid polypeptide of said second AAV comprises a glycine, leucine, and serine (GLS) sequence immediately following said amino acid tag.
Embodiment 64. The composition of any one of embodiments 50-63, wherein said capsid polypeptide of said second AAV comprises a protease cleavage site.
Embodiment 65. The composition of any one of embodiments 56-57, wherein said capsid polypeptide of said second AAV comprises a protease cleavage site.
Embodiment 66. The composition of embodiment 65, wherein said protease cleavage site of said second AAV immediately precedes said amino acid tag or precedes said amino acid tag by no more than 20 amino acid residues.
Embodiment 67. The composition of embodiment 65, wherein said protease cleavage site of said second AAV precedes said amino acid tag by no more than ten amino acid residues.
Embodiment 68. The composition of embodiment 65, wherein said protease cleavage site of said second AAV immediately follows said amino acid tag or follows said amino acid tag by no more than 20 amino acid residues.
Embodiment 69. The composition of embodiment 65, wherein said protease cleavage site of said second AAV follows said amino acid tag by no more than ten amino acid residues.
Embodiment 70. The composition of embodiment 65, wherein said capsid polypeptide of said second AAV comprises an N-terminal linker immediately preceding said protease cleavage site or preceding said protease cleavage site by no more than ten amino acid residues, wherein said protease cleavage site immediately precedes said amino acid tag or precedes said amino acid tag by no more than ten amino acid residues.
Embodiment 71. The composition of embodiment 70, wherein said N-terminal linker comprises (a) a leucine and alanine (LA) sequence or (b) a threonine and glycine (TG) sequence.
Embodiment 72. The composition of any one of embodiments 65-71, wherein said capsid polypeptide of said second AAV comprises a C-terminal linker immediately following said amino acid tag or following said amino acid tag by no more than ten amino acid residues.
Embodiment 73. The composition of embodiment 72, wherein said C-terminal linker comprises (a) an alanine (A) or (b) a glycine, leucine, and serine (GLS) sequence.
Embodiment 74. The composition of embodiment 65, wherein said capsid polypeptide of said second AAV comprises a leucine and alanine (LA) sequence immediately preceding said protease cleavage site, wherein said protease cleavage site immediately precedes said amino acid tag, and wherein an alanine immediately follows said amino acid tag.
Embodiment 75. The composition of embodiment 65, wherein said capsid polypeptide of said second AAV comprises a threonine and glycine (TG) sequence immediately preceding said protease cleavage site, wherein said protease cleavage site immediately precedes said amino acid tag, and wherein a glycine, leucine, and serine (GLS) sequence immediately follows said amino acid tag.
Embodiment 76. The composition of embodiment 65, wherein said capsid polypeptide of said second AAV comprises an N-terminal linker immediately preceding said amino acid tag or preceding said amino acid tag by no more than ten amino acid residues, wherein said protease cleavage site immediately follows said amino acid tag or follows said amino acid tag by no more than ten amino acid residues.
Embodiment 77. The composition of embodiment 76, wherein said N-terminal linker comprises (a) a leucine and alanine (LA) sequence or (b) a threonine and glycine (TG) sequence.
Embodiment 78. The composition of any one of embodiments 76-77, wherein said capsid polypeptide of said second AAV comprises a C-terminal linker immediately following said protease cleavage site or following said protease cleavage site by no more than ten amino acid residues.
Embodiment 79. The composition of embodiment 78, wherein said C-terminal linker comprises (a) an alanine (A) or (b) a glycine, leucine, and serine (GLS) sequence.
Embodiment 80. The composition of embodiment 65, wherein said capsid polypeptide of said second AAV comprises a leucine and alanine (LA) sequence immediately preceding said amino acid tag, wherein said protease cleavage site immediately follows said amino acid tag, and wherein an alanine immediately follows said protease cleavage site.
Embodiment 81. The composition of embodiment 65, wherein said capsid polypeptide of said second AAV comprises a threonine and glycine (TG) sequence immediately preceding said amino acid tag, wherein said protease cleavage site immediately follows said amino acid tag, and wherein a glycine, leucine, and serine (GLS) sequence immediately follows said protease cleavage site.
Embodiment 82. The composition of embodiment 65, wherein said capsid polypeptide of said second AAV comprises a C-terminal linker immediately following said protease cleavage site or following said protease cleavage site by no more than ten amino acid residues, and wherein said protease cleavage site immediately follows said amino acid tag or follows said amino acid tag by no more than ten amino acid residues.
Embodiment 83. The composition of embodiment 82, wherein said C-terminal linker comprises (a) an alanine (A) or (b) a glycine, leucine, and serine (GLS) sequence.
Embodiment 84. The composition of any one of embodiments 82-83, wherein said capsid polypeptide of said second AAV comprises an N-terminal linker immediately preceding said amino acid tag or preceding said amino acid tag by no more than ten amino acid residues.
Embodiment 85. The composition of embodiment 84, wherein said N-terminal linker comprises (a) a leucine and alanine (LA) sequence or (b) a threonine and glycine (TG) sequence.
Embodiment 86. The composition of any one of embodiments 65-85, wherein said protease cleavage site is a cleavage site for a protease selected from the group consisting of a TEV protease, a PreScission protease, thrombin, Factor Xa, and enterokinase.
Embodiment 87. The composition of any one of embodiments 65-85, wherein said protease cleavage site comprises the amino acid sequence set forth in any one of SEQ ID NOs:20-25.
Embodiment 88. The composition of any one of embodiments 50-87, wherein said second AAV is an AAV2.
Embodiment 89. The composition of any one of embodiments 50-88, wherein said composition comprises a conjugating polypeptide having the ability to bind covalently to said amino acid segment of said first AAV.
Embodiment 90. The composition of embodiment 89, wherein said conjugating polypeptide comprises a first binding motif
Embodiment 91. The composition of embodiment 90, wherein said first binding motif comprises (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27.
Embodiment 92. The composition of embodiment 91, wherein said first binding motif comprises the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.
Embodiment 93. The composition of embodiment 91, wherein said first binding motif comprises the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.
Embodiment 94. The composition of embodiment 91, wherein said first binding motif comprises the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.
Embodiment 95. The composition of embodiment 94, wherein said first binding motif comprises the amino acid sequence set forth in SEQ ID NO:11.
Embodiment 96. The composition of embodiment 91, wherein said first binding motif comprises the amino acid sequence set forth in SEQ ID NO:10, SEQ ID NO:26, or SEQ ID NO:27.
Embodiment 97. The composition of embodiment 90, wherein said first binding motif comprising (i) the amino acid sequence set forth in SEQ ID NO:12 or (ii) the amino acid sequence set forth in SEQ ID NO:12 with 30 or less amino acid additions, deletions, or substitutions with the proviso that the amino acid located at a position corresponding to position 31 of SEQ ID NO:12 remains a lysine, with the proviso that the amino acid located at a position corresponding to position 77 of SEQ ID NO:12 remains a glutamic acid, and with the proviso that the amino acid located at a position corresponding to position 117 of SEQ ID NO:12 remains an aspartic acid.
Embodiment 98. The composition of embodiment 97, wherein said first binding motif comprises the amino acid sequence set forth in SEQ ID NO:12 with 25 or less amino acid additions, deletions, or substitutions with the proviso that the amino acid located at a position corresponding to position 31 of SEQ ID NO:12 remains a lysine, with the proviso that the amino acid located at a position corresponding to position 77 of SEQ ID NO:12 remains a glutamic acid, and with the proviso that the amino acid located at a position corresponding to position 117 of SEQ ID NO:12 remains an aspartic acid.
Embodiment 99. The composition of embodiment 97, wherein said first binding motif comprises the amino acid sequence set forth in SEQ ID NO:12 with 20 or less amino acid additions, deletions, or substitutions with the proviso that the amino acid located at a position corresponding to position 31 of SEQ ID NO:12 remains a lysine, with the proviso that the amino acid located at a position corresponding to position 77 of SEQ ID NO:12 remains a glutamic acid, and with the proviso that the amino acid located at a position corresponding to position 117 of SEQ ID NO:12 remains an aspartic acid.
Embodiment 100. The composition of embodiment 99, wherein said first binding motif comprises the amino acid sequence set forth in SEQ ID NO:13.
Embodiment 101. The composition of embodiment 97, wherein said first binding motif comprises the amino acid sequence set forth in SEQ ID NO:12.
Embodiment 102. The composition of embodiment 90, wherein said first binding motif comprising the amino acid sequence set forth in any one of: SEQ ID NO:14, SEQ ID NO:15, SEQ ID NO:16, and SEQ ID NO:17.
Embodiment 103. The composition of any one of embodiments 89-102, wherein said composition comprises said conjugating polypeptide covalently attached to said amino acid segment of said first AAV.
Embodiment 104. The composition of any one of embodiments 89-103, wherein said conjugating polypeptide comprises the ability to bind covalently to said amino acid tag of said second AAV.
Embodiment 105. The composition of any one of embodiments 89-104, wherein said composition comprises said conjugating polypeptide covalently attached to said amino acid segment of said first AAV and to said amino acid tag of said second AAV.
Embodiment 106. The composition of any one of embodiments 90-105, wherein said conjugating polypeptide comprises two of said first binding motifs of any of embodiments 90-102.
Embodiment 107. The composition of embodiment 106, wherein the amino acid sequence of each of said two is identical.
Embodiment 108. The composition of embodiment 106, wherein the amino acid sequence of each of said two is different.
Embodiment 109. The composition of any one of embodiments 106-108, wherein said two of said first binding motifs are separated by a linker.
Embodiment 110. The composition of embodiment 109, wherein said linker comprises the amino acid sequence set forth in SEQ ID NO:41, SEQ ID NO:42, or SEQ ID NO:43.
Embodiment 111. The composition of any one of embodiments 90-105, wherein said conjugating polypeptide comprises a second binding motif.
Embodiment 112. The composition of embodiment 111, wherein said second binding motif comprises the ability to bind covalently to said amino acid tag of said second AAV.
Embodiment 113. The composition of any one of embodiments 111-112, wherein said second binding motif comprises:

(a) the amino acid sequence set forth in SEQ ID NO:18 or the amino acid sequence set forth in SEQ ID NO:18 with 25 or less amino acid additions, deletions, or substitutions,

(b) the amino acid sequence set forth in SEQ ID NO:19 or the amino acid sequence set forth in SEQ ID NO:19 with 25 or less amino acid additions, deletions, or substitutions, or

(c) the amino acid sequence set forth in SEQ ID NO:45 or the amino acid sequence set forth in SEQ ID NO:45 with 25 or less amino acid additions, deletions, or substitutions.

Embodiment 114. The composition of any one of embodiments 111-113, wherein said first binding motif and said second binding motif are separated by a linker.
Embodiment 115. The composition of embodiment 114, wherein said linker comprises the amino acid sequence set forth in SEQ ID NO:41, SEQ ID NO:42, or SEQ ID NO:43.
Embodiment 116. The composition of any one of embodiments 89-115, wherein said conjugating polypeptide comprises SEQ ID NO:41.
Embodiment 117. A conjugating polypeptide comprising (a) a first binding motif and (b) a second binding motif, wherein said first binding motif comprises (i) the amino acid sequence set forth in SEQ ID NO:10 (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27.
Embodiment 118. The conjugating polypeptide of embodiment 117, wherein said first binding motif comprises the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.
Embodiment 119. The conjugating polypeptide of embodiment 117, wherein said first binding motif comprises the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.
Embodiment 120. The conjugating polypeptide of embodiment 117, wherein said first binding motif comprises the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.
Embodiment 121. The conjugating polypeptide of embodiment 120, wherein said first binding motif comprises the amino acid sequence set forth in SEQ ID NO:11.
Embodiment 122. The conjugating polypeptide of embodiment 117, wherein said first binding motif comprises the amino acid sequence set forth in SEQ ID NO:10, SEQ ID NO:26, or SEQ ID NO:27.
Embodiment 123. The conjugating polypeptide of any one of embodiments 117-122, wherein said second binding motif comprises (i) the amino acid sequence set forth in SEQ ID NO:10, (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27.
Embodiment 124. The conjugating polypeptide of embodiment 123, wherein said second binding motif comprises the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.
Embodiment 125. The conjugating polypeptide of embodiment 123, wherein said second binding motif comprises the amino acid sequence set forth in SEQ ID NO:10 with 15 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.
Embodiment 126. The conjugating polypeptide of embodiment 123, wherein said second binding motif comprises the amino acid sequence set forth in SEQ ID NO:10 with 11 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid.
Embodiment 127. The conjugating polypeptide of embodiment 126, wherein said second binding motif comprises the amino acid sequence set forth in SEQ ID NO:11.
Embodiment 128. The conjugating polypeptide of embodiment 123, wherein said second binding motif comprises the amino acid sequence set forth in SEQ ID NO:10, SEQ ID NO:26, SEQ ID NO:27.
Embodiment 129. The conjugating polypeptide of any one of embodiments 117-122, wherein said second binding motif comprises:

(a) the amino acid sequence set forth in SEQ ID NO:18 or the amino acid sequence set forth in SEQ ID NO:18 with 25 or less amino acid additions, deletions, or substitutions,

(b) the amino acid sequence set forth in SEQ ID NO:19 or the amino acid sequence set forth in SEQ ID NO:19 with 25 or less amino acid additions, deletions, or substitutions, or

(c) the amino acid sequence set forth in SEQ ID NO:45 or the amino acid sequence set forth in SEQ ID NO:45 with 25 or less amino acid additions, deletions, or substitutions.

Embodiment 130. The conjugating polypeptide of any one of embodiments 117-122, wherein said first binding motif and said second binding motif are separated by a linker.
Embodiment 131. The conjugating polypeptide of embodiment 130, wherein said linker comprises the amino acid sequence set forth in SEQ ID NO:41, SEQ ID NO:42, or SEQ ID NO:43.
Embodiment 132. The conjugating polypeptide of any one of embodiments 117-131, wherein said conjugating polypeptide comprises SEQ ID NO:41.
Embodiment 133. An isolated nucleic acid comprising a nucleic acid sequence encoding a conjugating polypeptide of any one of embodiments 117-132.
Embodiment 134. A method for producing a composition comprising a first Adeno-Associated Virus (AAV) covalently linked to a second AAV, wherein said first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, wherein said second AAV comprises a capsid polypeptide comprising an amino acid tag, wherein said method comprises:

(a) contacting said first AAV with a conjugating polypeptide to form a first AAV-conjugating polypeptide complex, wherein said conjugating polypeptide comprises a first binding motif having the ability to bind covalently to said amino acid segment of said first AAV and a second binding motif having the ability to bind covalently to said amino acid tag of said second AAV, and

(b) contacting said first AAV-conjugating polypeptide complex with said second AAV for form a first AAV-conjugating polypeptide-second AAV complex, thereby producing said composition comprising said first AAV covalently linked to said second AAV.

Embodiment 135. A method for producing a composition comprising a first Adeno-Associated Virus (AAV) covalently linked to a second AAV, wherein said first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, wherein said second AAV comprises a capsid polypeptide comprising an amino acid tag, wherein said method comprises:

(a) contacting said second AAV with a conjugating polypeptide to form a second AAV-conjugating polypeptide complex, wherein said conjugating polypeptide comprises a first binding motif having the ability to bind covalently to said amino acid segment of said first AAV and a second binding motif having the ability to bind covalently to said amino acid tag of said second AAV, and

(b) contacting said second AAV-conjugating polypeptide complex with said first AAV for form a second AAV-conjugating polypeptide-first AAV complex, thereby producing said composition comprising said first AAV covalently linked to said second AAV.

Embodiment 136. The method of any one of embodiments 134-135, wherein said contacting step with said first AAV comprises using a solution comprising a titer of said AAV that is greater than 10¹⁴.
Embodiment 137. The method of any one of embodiments 134-136, wherein said first AAV is an AAV of any one of embodiments 1-46.
Embodiment 138. The method of any one of embodiments 134-137, wherein said second AAV is the second AAV according to any one of embodiments 56-88.
Embodiment 139. The method of any one of embodiments 134-138, wherein said conjugating polypeptide is a conjugating polypeptide according to any one of embodiments 117-132.
Embodiment 140. The method of any one of embodiments 134-139, wherein said method comprises performing a purification procedure (i) after said step (a) of embodiment 134 to form a composition enriched for said first AAV-conjugating polypeptide complex, wherein said composition enriched for said first AAV-conjugating polypeptide complex is used in said step (b) of embodiment 134 or (ii) after said step (a) of embodiment 135 to form a composition enriched for said second AAV-conjugating polypeptide complex, wherein said composition enriched for said second AAV-conjugating polypeptide complex is used in said step (b) of embodiment 135.
Embodiment 141. The method of any one of embodiments 134-140, wherein said method comprises performing a purification procedure (i) after said step (b) of embodiment 134 to form a composition enriched for said first AAV-conjugating polypeptide-second AAV complex or (ii) after said step (b) of embodiment 135 to form a composition enriched for said second AAV-conjugating polypeptide-first AAV complex.

Embodiment 142. A method for producing a composition comprising a first Adeno-Associated Virus (AAV) covalently linked to a second AAV, wherein said first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, wherein said second AAV comprises a capsid polypeptide comprising an amino acid tag, wherein said method comprises:
(a) contacting said first AAV with a conjugating polypeptide to form a first AAV-conjugating polypeptide complex, wherein said conjugating polypeptide comprises a first binding motif having the ability to bind covalently to said amino acid segment of said first AAV and a second binding motif having the ability to bind covalently to said amino acid tag of said second AAV, and

(b) contacting said first AAV-conjugating polypeptide complex with said second AAV for form a first AAV-conjugating polypeptide-second AAV complex, thereby producing said composition comprising said first AAV covalently linked to said second AAV,

wherein (i) said capsid polypeptide of said first AAV comprises a protease cleavage site within about 20 amino acid residues of said amino acid segment and said method comprises contacting said first AAV with a protease for said protease cleavage site of said first AAV prior to or during said step (a), (ii) said capsid polypeptide of said second AAV comprises a protease cleavage site within about 20 amino acid residues of said amino acid tag and said method comprises contacting said second AAV with a protease for said protease cleavage site of said second AAV prior to or during said step (b), or (iii) said capsid polypeptide of said first AAV comprises a protease cleavage site within about 20 amino acid residues of said amino acid segment, said method comprises contacting said first AAV with a protease for said protease cleavage site of said first AAV prior to or during said step (a), said capsid polypeptide of said second AAV comprises a protease cleavage site within about 20 amino acid residues of said amino acid tag, and said method comprises contacting said second AAV with a protease for said protease cleavage site of said second AAV prior to or during said step (b).

Embodiment 143. A method for producing a composition comprising a first Adeno-Associated Virus (AAV) covalently linked to a second AAV, wherein said first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, wherein said second AAV comprises a capsid polypeptide comprising an amino acid tag, wherein said method comprises:

(a) contacting said second AAV with a conjugating polypeptide to form a second AAV-conjugating polypeptide complex, wherein said conjugating polypeptide comprises a first binding motif having the ability to bind covalently to said amino acid segment of said first AAV and a second binding motif having the ability to bind covalently to said amino acid tag of said second AAV, and

(b) contacting said second AAV-conjugating polypeptide complex with said first AAV for form a second AAV-conjugating polypeptide-first AAV complex, thereby producing said composition comprising said first AAV covalently linked to said second AAV,

wherein (i) said capsid polypeptide of said first AAV comprises a protease cleavage site within about 20 amino acid residues of said amino acid segment and said method comprises contacting said first AAV with a protease for said protease cleavage site of said first AAV prior to or during said step (b), (ii) said capsid polypeptide of said second AAV comprises a protease cleavage site within about 20 amino acid residues of said amino acid tag and said method comprises contacting said second AAV with a protease for said protease cleavage site of said second AAV prior to or during said step (a), or (iii) said capsid polypeptide of said first AAV comprises a protease cleavage site within about 20 amino acid residues of said amino acid segment, said method comprises contacting said first AAV with a protease for said protease cleavage site of said first AAV prior to or during said step (b), said capsid polypeptide of said second AAV comprises a protease cleavage site within about 20 amino acid residues of said amino acid tag, and said method comprises contacting said second AAV with a protease for said protease cleavage site of said second AAV prior to or during said step (a).

Embodiment 144. The method of any one of embodiments 142-143, wherein said contacting step with said first AAV comprises using a solution comprising a titer of said AAV that is greater than 10¹⁴.
Embodiment 145. The method of any one of embodiments 142-144, wherein said first AAV is an AAV of any one of embodiments 1-46.
Embodiment 146. The method of any one of embodiments 142-145, wherein said second AAV is the second AAV according to any one of embodiments 56-88.
Embodiment 147. The method of any one of embodiments 142-146, wherein said conjugating polypeptide is a conjugating polypeptide according to any one of embodiments 117-132.
Embodiment 148. The method of any one of embodiments 142-147, wherein said method comprises performing a purification procedure (i) after said step (a) of embodiment 142 to form a composition enriched for said first AAV-conjugating polypeptide complex, wherein said composition enriched for said first AAV-conjugating polypeptide complex is used in said step (b) of embodiment 142 or (ii) after said step (a) of embodiment 143 to form a composition enriched for said second AAV-conjugating polypeptide complex, wherein said composition enriched for said second AAV-conjugating polypeptide complex is used in said step (b) of embodiment 143.
Embodiment 149. The method of any one of embodiments 142-148, wherein said method comprises performing a purification procedure (i) after said step (b) of embodiment 142 to form a composition enriched for said first AAV-conjugating polypeptide-second AAV complex or (ii) after said step (b) of embodiment 143 to form a composition enriched for said second AAV-conjugating polypeptide-first AAV complex.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An Adeno-Associated Virus (AAV) comprising a capsid polypeptide,
wherein said capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions.

2. The AAV of claim 1, wherein said AAV is an AAV2.

3. The AAV of claim 1, wherein said amino acid segment comprises the amino acid sequence set forth in SEQ ID NO:1.

4. The AAV of claim 1, wherein said capsid polypeptide comprises said amino acid segment at an amino acid position within three amino acid residues of an amino acid residue corresponding to position 453 or position 588 of a reference VP1 polypeptide having the amino acid sequence set forth in SEQ ID NO:2.

5. The AAV of claim 1, wherein said capsid polypeptide comprises a protease cleavage site.

6-8. (canceled)

9. A composition comprising a first Adeno-Associated Virus (AAV) and a second AAV genetically different from said first AAV, wherein said first AAV is an AAV comprising a capsid polypeptide, wherein said capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions.

10. The composition of claim 9, wherein said first and second AAVs are covalently attached to each other.

11. The composition of claim 10, wherein said first AAV is covalently attached to said second AAV via at least one covalent bond of said amino acid segment of said first AAV.

12. The composition of claim 9, wherein the capsid polypeptides of said first and second AAVs are identical.

13. The composition of claim 9, wherein the capsid polypeptides of said first and second AAVs are different.

14. The composition of claim 13, wherein the capsid polypeptides of said second AAV lacks said amino acid segment.

15. A conjugating polypeptide comprising (a) a first binding motif and (b) a second binding motif, wherein said first binding motif comprises (i) the amino acid sequence set forth in SEQ ID NO:10 (ii) the amino acid sequence set forth in SEQ ID NO:10 with 20 or less amino acid additions, deletions, or substitutions with the proviso that the amino acid located at a position corresponding to position 10 of SEQ ID NO:10 remains a lysine and with the proviso that the amino acid located at a position corresponding to position 56 of SEQ ID NO:10 remains a glutamic acid, (iii) the amino acid sequence set forth in SEQ ID NO:26, or (iv) the amino acid sequence set forth in SEQ ID NO:27.

16. A method for producing a composition comprising a first Adeno-Associated Virus (AAV) covalently linked to a second AAV, wherein said first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, wherein said second AAV comprises a capsid polypeptide comprising an amino acid tag, wherein said method comprises:
(a) contacting said first AAV with a conjugating polypeptide to form a first AAV-conjugating polypeptide complex, wherein said conjugating polypeptide comprises a first binding motif having the ability to bind covalently to said amino acid segment of said first AAV and a second binding motif having the ability to bind covalently to said amino acid tag of said second AAV, and
(b) contacting said first AAV-conjugating polypeptide complex with said second AAV for form a first AAV-conjugating polypeptide-second AAV complex, thereby producing said composition comprising said first AAV covalently linked to said second AAV.

17. A method for producing a composition comprising a first Adeno-Associated Virus (AAV) covalently linked to a second AAV, wherein said first AAV comprises a capsid polypeptide comprising an amino acid segment comprising (i) the amino acid sequence set forth in SEQ ID NO:1 or (ii) the amino acid sequence set forth in SEQ ID NO:1 with one, two, or three amino acid substitutions, wherein said second AAV comprises a capsid polypeptide comprising an amino acid tag, wherein said method comprises:
(a) contacting said second AAV with a conjugating polypeptide to form a second AAV-conjugating polypeptide complex, wherein said conjugating polypeptide comprises a first binding motif having the ability to bind covalently to said amino acid segment of said first AAV and a second binding motif having the ability to bind covalently to said amino acid tag of said second AAV, and
(b) contacting said second AAV-conjugating polypeptide complex with said first AAV for form a second AAV-conjugating polypeptide-first AAV complex, thereby producing said composition comprising said first AAV covalently linked to said second AAV.

18. The method of claim 16, wherein said contacting step with said first AAV comprises using a solution comprising a titer of said AAV that is greater than 10¹⁴.

19. The method of claim 16, wherein said first AAV is an AAV comprising a capsid polypeptide, wherein said capsid polypeptide comprises an amino acid segment comprising (i) the amino acid sequence set forth in SEQ ID NO:1 or (ii) the amino acid sequence set forth in SEQ ID NO:1 with one, two, or three amino acid substitutions.

20-23. (canceled)
